(19) United States
(12) Patent Application Publication
(10) Pub. No.: US 2011/0124831 A1
(43) Pub. Date: May 26, 2011

(54) SELF-ASSEMBLED OLEFIN POLYMERIZATION CATALYST

(75) Inventor: He-Kuan Luo, Singapore (SG)

(73) Assignee: AGENCY FOR SCIENCE, TECHNOLOGY AND RESEARCH, Singapore (SG)

(21) Appl. No.: 12/812,942

(22) PCT Filed: Jan. 14, 2008
(86) PCT No.: PCT/SG08/00015

§ 371 Date: Jan. 18, 2011

(51) Int. Cl. C08F 4/76; C07F 7/28; C07C 251/24; C07F 7/00; B01J 31/12; B01J 31/14

(52) U.S. Cl. 526/172; 556/56; 564/270; 502/167; 502/103; 502/117; 502/134

(57) ABSTRACT

The present invention relates to a self-assembled olefin polymerization catalyst comprising a transition metal compound according to formula (I) L_{q}MₘXₙ wherein M is a transition metal selected from the group consisting of Group 3-11 of the periodic table; X is independently selected from the group consisting of H, halogen, CN, optionally substituted N(R^{a})₂, OH, optionally substituted C₁-C₂₀ alkyl, optionally substituted C₁-C₂₀ alkoxy, wherein R^{a} is independently selected from the group consisting of optionally substituted C₁-C₂₀ alkyl, optionally substituted C₆-C₂₀ aryl and halogen; q is an integer of at least 2; m is an integer of at least 2; n is an integer making (I) electrically neutral; L is independently a ligand which has at least two linked coordination units, wherein each coordination unit binds to a different transition metal. The present invention also relates to a process for the polymerization of olefins using the transition metal compound of the invention and to the polyolefins obtained from this polymerization process. Finally, the invention also relates to new ligands L present in the transition metal compound and to methods of making the ligand L.

### FIELD OF THE INVENTION

The present invention relates to a self-assembled olefin polymerization catalyst, to a process for the polymerization of olefins and to the polyolefins obtained therefrom. The present invention also relates to a compound constituting a ligand system which builds the self-assembled olefin polymerization catalyst and a preparation process thereof.

### BACKGROUND

The production of polyolefins is a very important branch of industry, as in 2005 about 100 million tons of polyolefins have been produced globally. The catalysts for olefin polymerization play a key role in the preparation process, resulting in the development of highly efficient olefin polymerization catalysts. Still, this is a very hot research area. After the traditional highly efficient multi-site Ziegler-Natta catalysts, such as TiClₙ/MgCl₂(n=3.4)^{[1]}, and single-site group-4 metallocene catalysts have been extensively studied and applied in industry^{[2]}, in the past decade much attention^{[3]} has been paid to non-cyclopentadienyl single-site catalysts using heteroatom coordination, such as N, O atoms that have attracted much interests. To date several highly efficient catalysts have been identified, such as α-diimine-Ni(II)/Pd(II)^{[4]}, 2,6-diiminopyridine-Fe(II)^{[5]}, phenoxy-imine-Ni^{[6]} and phenoxy-imine-Ti/Zr catalysts^{[7]} (see FIG. 1).

Among the non-cyclopentadienyl catalysts, phenoxy-imine-based Group 4 catalysts^{[7]} (see FIG. 2 and Model-1 in FIG. 3) have received much attention in both academia and industry because they intrinsically have high activity and the same group metal. Group 4 based traditional highly efficient Ziegler-Natta catalysts and single-site Group-4 metallocene catalysts have been successfully applied in industry. However, this kind of non-cyclopentadienyl catalysts have limited lifetime predominantly because of the transfer of supporting ligand to aluminum in the co-catalyst mixture (see FIG. 4)^{[8c,8d]}, especially under elevated temperatures used in industry. In some cases the catalysts decay quickly even within several minutes. As a consequence these catalysts were usually studied under low temperature (such as room temperature) and/or short reaction time even between 1-15 minutes^{[7]}. This greatly hinders the application of this kind of catalysts in industry.

As titanium and zirconium catalysts based on phenoxy-imine ligand (see FIG. 2) have limited lifetime, much effort has been made to solve this problem using tetradentate ligands, which were expected to form more stable catalysts of coordination model-2 (see scheme 3). Fujita^{[7j]} and co-workers have investigated tetradentate ligands of Cₙ-chain-bridged phenoxy-imine units (n=2-6; see FIG. 5) forming model-2 catalyst (see scheme 3), the results showed that the ligands of longer bridge (n=5 or 6) displayed high activity for five minutes run, while the ligands of shorter bridge (n=2-4) displayed very low activity, and the issue of catalyst rapid deactivation was not addressed.

Gibson and Scott have indicated that phenoxy-imine-Ti/Zr catalysts have limited lifetime although their initial activity is quite high within 5 minutes. They also believed that the tetradentate ligands incorporating titanium and zirconium may form more stable catalysts bearing the coordination model-2 shown in FIG. 3 with two imine-N linked^{[8]}. However experimental results demonstrated that the tetradentate ligands III and XII (see FIG. 6) did not afford olefin polymerization catalysts, principally because of a destructive 1,2-migratory insertion of a metal-bound alkyl/polymeryl chain into the imine C═N unit^{[8a-8c]}. Subsequently, Scott and co-workers^{[8b,8c]} found that introducing an alkyl group at the position R⁴ (see ligand XI in FIG. 6) of a zirconium salicylaldiminato complex leads to a long-lived catalyst (1 hour test in toluene) for ethylene polymerization because of steric blocking of an intramolecular 1,2-migratory insertion, but this steric blocking promotes a new radical catalyst decomposition mechanism in certain instances, thus resulting in far lower activity compared to the corresponding catalyst based on ligand I. In addition, all the zirconium complexes of ligands IV-X have no activity probably due to the lack of steric bulk in the phenolate 2-position^{[8c]}. In their further studies, Gibson and Scott^{[8d]} investigated more tetradentate ligands (see XIII-XVII in FIG. 6). For titanium complexes, [(XIII) TiCl₂] had no activity for ethylene polymerization when treated with MAO because the two chloride ligands are in trans-arrangement. The cis-complex [(XIV) TiCl₂] however was also unproductive, perhaps due to enhanced imine reactivity brought on by ring-strain in the diamine backbone. Complex [(XV) TiCl₂] produced only a trace of polymer. Although complexes [(XVI) TiCl₂] and [(XVII) TiCl₂] demonstrated significantly improved activity at 25° C. in excess of 2×10³ Kg_{PE} mol_{cat}⁻¹ h⁻¹ bar⁻¹ (1 hour test in toluene), the overall productivities are rather lower at 50° C. resulting from more rapid catalyst decomposition. For zirconium complexes, complex [(XV) ZrCl₂] produced only a trace of polymer, the complexes [(XVI) ZrCl₂] and [(XVII) ZrCl₂] demonstrated only low activities.

Therefore, after various tetradentate ligands have been investigated, the challenge to develop long-lived highly efficient non-cyclopentadienyl catalysts is still remained. Thus, a catalyst is desired which, departing from the above common idea and using a different strategy, has an increased lifetime, a higher activity and affording polymers with higher molecular weight.

### SUMMARY

The present invention has been developed on the afore-mentioned background.

In a first aspect, the present invention provides a self-assembled olefin polymerization catalyst comprising a transition metal compound according to formula (I)

L_{q}MₘXₙ (I)

wherein

M is a transition metal selected from the group consisting of Group 3-11 of the periodic table;

X is independently selected from the group consisting of H, halogen, CN, optionally substituted N(R^{a})₂, OH, optionally substituted C₁-C₂₀ alkyl, optionally substituted C₁₋₂₀ alkoxy, wherein R^{a} is independently selected from the group consisting of optionally substituted C₁-C₂₀ alkyl, optionally substituted C₆-C₂₀ aryl and halogen;

q is an integer of at least 2;

m is an integer of at least 2;

n is an integer making (I) electrically neutral;

L is independently a ligand which has at least two linked coordination units, wherein each coordination unit binds to a different transition metal.

In a second aspect, the present invention provides a process for polymerization or copolymerization of an olefin or a mixture of olefins in the presence of the self-assembled olefin polymerization catalyst described in the present invention.

In a third aspect, the present invention provides polyolefins obtainable according to the process of the present invention.

In a fourth aspect, the present invention provides a compound (also referred to herein as ligand) according to the following formula (II)

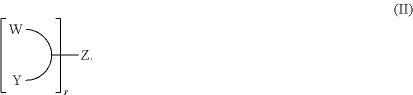

In a fifth aspect, the present invention provides a process for producing a ligand of the invention by Schiff-Base condensation between an aldehyde or ketone with a di-aniline, tri-aniline or tetrakis-aniline.

In a sixth aspect, the present invention provides a process for producing a ligand of the invention by Schiff-Base condensation between an aniline and a di-aldehyde/di-ketone, tri-aldehyde/tri-ketone or tetrakis-aldehyde/tetrakis-ketone.

### BRIEF DESCRIPTION OF THE DRAWINGS

FIG. 1 illustrates examples of non-cyclopentadienyl single-site catalysts according to the state of the art.

FIG. 2 illustrates representative titanium and zirconium catalysts of the prior art based on phenoxy-imine ligand bearing coordination model-1 as shown in FIG. 3.

FIG. 3 illustrates a comparison of three possible coordination models for a catalyst, wherein model-1 and model-2 are state of the art and model-3 illustrates one of the possible coordination models of the present invention.

FIG. 4 illustrates a scheme showing the ligand transfer from the catalyst to the aluminum atom of the co-catalyst. This is one of the reasons for the limited lifetime of the catalysts of the prior art.

FIG. 5 illustrates a tetradentate ligand forming model-2 type catalyst as shown in FIG. 3.

FIG. 6 illustrates further tetradentate ligands forming model-2 type catalyst as shown in FIG. 3.

FIG. 7 illustrates the self-assembling strategy of the present invention in order to synthesize olefin polymerization catalysts. It is shown that because of its size, length and angle, the bridging spacer makes the two units of one inventive olefin polymerization catalyst to coordinate with two different metal atoms. The self-assembling achieves long-lived highly efficient polymerization catalysts.

FIG. 8a illustrates one of the possible self-assembled catalyst structures of the present invention. In this case the self-assembled structure is a linear assembling structure which may further form macrocycles of any size.

FIG. 8b illustrates a further possibility for the self-assembled catalyst structures of the present invention. In this particular case the self-assembled structure is a macrocyclic assembling structure having at least 6 metal centres.

FIG. 9 illustrates a possible synthesis route for bis-phenoxy-imine and self-assembled catalysts. In the compound (XVIII) the arrow indicates that the distance between the two coordination sites is too long for coordination of one and the same metal atom, therefore the second NO unit will coordinate with a second metal atom to form the self-assembled catalyst.

FIG. 10 illustrates several compounds for the comparison of self-assembled multi-nuclear catalysts (SA-Ti-1, SA-Zr) with known catalysts (Known-Ti, Known-Zr).

FIG. 11 is a graph illustrating a comparison of the activities of SA-Ti-1 and Known-Ti in three reaction periods.

FIG. 12 is a graph illustrating a comparison of the activities of SA-Zr and Known-Zr in five reaction periods.

FIG. 13 illustrates the amounts of polymer produced after several reaction times using SA-Ti-1 and Known-Ti catalysts. It is shown that for SA-Ti-1 the amount of polyethylene increased quickly with the prolongation of reaction time, while for Known-Ti the amount of polyethylene increased very slowly.

FIG. 14 illustrates the amounts of polymer produced after several reaction times using SA-Zr and Known-Zr catalysts. It is shown that for SA-Zr the amount of polyethylene increased quickly with the prolongation of reaction time, while for Known-Zr the amount of polyethylene is almost the same with different reaction times.

FIG. 15 illustrates the synthesis of bis-phenoxy-imine ligand (XIX) and the corresponding self-assembled catalyst (SA-Ti-2).

FIG. 16 illustrates the molecular structure of bis-phenoxy-imine ligand (XIX) obtained by single crystal X-Ray diffraction. This X-Ray structure clearly shows that the distance between the two coordination sites is too long for coordination of one and the same metal atom, therefore the second NO unit will coordinate with a second metal atom to form the self-assembled catalyst.

FIG. 17 is a graph illustrating a comparison of the activities of SA-Ti-2 and Known-Ti in three reaction periods.

FIG. 18 illustrates the amounts of polymer produced after several reaction times using SA-Ti-2 and Known-Ti catalysts. It is shown that for SA-Ti-2 the amount of polyethylene increased quickly with the prolongation of reaction time, while for Known-Ti the amount of polyethylene increased very slowly.

FIG. 19 illustrates the reactor fouling after 2-hour ethylene polymerization using the SA-Ti-2 catalyst of the present invention and the Known-Ti catalysts. It is shown that the Known-Ti catalyst caused significant fouling, while the SA-Ti-2 catalyst only caused negligible fouling and the reactor was still clean after the polymerization reaction.

FIGS. 20a to 20c illustrate reaction schemes for one of the possibilities to prepare the self-assembled transition metal catalyst of the invention.

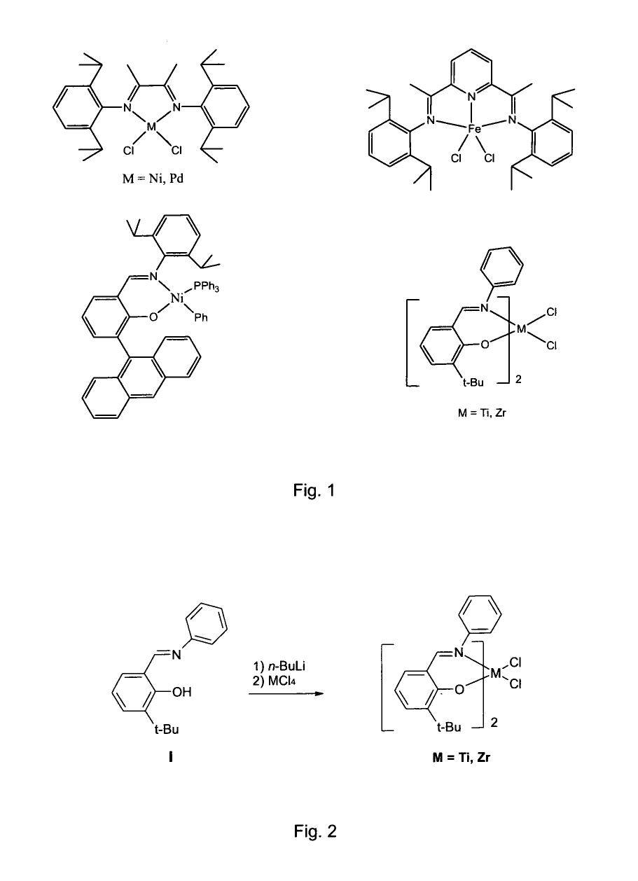

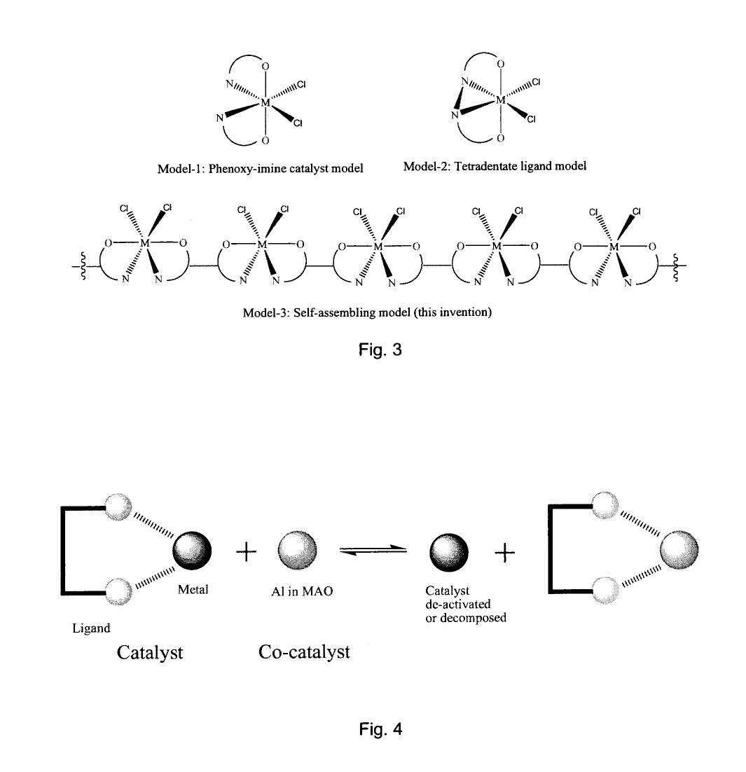

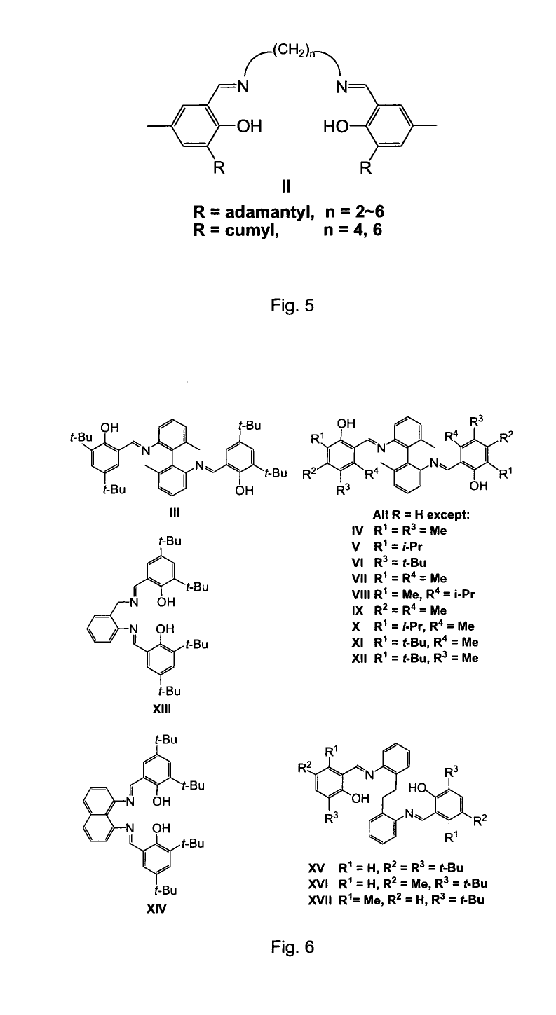

### DETAILED DESCRIPTION OF THE INVENTION

In the following description non-limiting embodiments of the process of the invention will be explained.

According to the present invention, it has been surprisingly found that a self-assembled olefin polymerization catalyst provides a long lifetime of the catalyst. The self-assembled olefin polymerization catalyst is also highly efficient in the polymerization of olefins and produces low molecular weight polyolefin polymers as well as high molecular weight polyolefin polymers. In addition, the self-assembled catalyst of the present invention is easy to prepare in a quantitative yield and with low costs.

Self-assembly (SA) is a term used to describe processes in which a disordered system of pre-existing components forms an organized structure or pattern as a consequence of specific, local interactions among the components themselves, without external direction. SA in the classic sense can be defined as the spontaneous and reversible organization of molecular units into ordered structures by non-covalent interactions. The first property of a self-assembled system that this definition suggests is the spontaneity of the self-assembly process: the interactions responsible for the formation of the self-assembled system act on a strictly local level—in other words, the nanostructure builds itself. Thus, SA is a very common phenomenon in chemistry that has been proved to be a tremendous tool to prepare highly efficient catalysts, such as chiral heterogeneous catalysts for asymmetric reactions^{[9]}, the catalyst is stable enough to be recycled for many times to achieve excellent activity and enantioselectivity. However this strategy has not been used to develop catalysts for olefin polymerization.

In the context of the present invention, the term “comprising” or “comprises” means including, but not limited to, whatever follows the word “comprising”. Thus, the use of the term “comprising” indicates that the listed elements are required or mandatory, but that other elements are optional and may or may not be present.

Therefore, the present invention provides a self-assembled olefin polymerization catalyst comprising a transition metal compound according to formula (I)

L_{q}MₘXₙ (I)

wherein

M is a transition metal selected from the group consisting of Group 3-11 of the periodic table;

X is independently selected from the group consisting of H, halogen, CN, optionally substituted N(R^{a})₂, OH and optionally substituted C₁-C₂₀ alkyl, optionally substituted C₁₋₂₀ alkoxy, wherein R^{a} is independently selected from the group consisting of optionally substituted C₁-C₂₀ alkyl, optionally substituted C₆-C₂₀ aryl and halogen;

q is an integer of at least 2;

m is an integer of at least 2;

n is an integer making (I) electrically neutral;

L is independently a ligand which has at least two linked coordination units, wherein each coordination unit binds to a different transition metal.

The transition metal M may be, but is not limited to, Sc, Y, La, Ti, Zr, Hf, V, Nb, Ta, Sm, Yb, Cr, Mo, W, Mn, Tc, Re, Fe, Ru, Os, Co, Rh, Ir, Ni, Pd, Pt, Cu, Zn or mixtures thereof. In one embodiment of the present invention, M may be Sc, Ti, Zr, Hf, V, Nb, Ta, Sm, Yb, Fe, Co, Rh, Ni or Pd, for example Ti, Zr, Hf, V, Nb, Ta, Sm, Yb or mixtures thereof. In a still further embodiment of the present invention, M may be Ti, Zr or mixtures thereof. The selection of the respective transition metal atom may depend on the reaction conditions and/or the olefin which should be polymerized.

The transition metal M may be in the oxidation state (O). Alternatively, in another embodiment the oxidation state of the transition metal may be between (I) and (VI) depending on the further type and number of the ligands. For example, M may represent a transition metal atom including, but not limited to, Sc(III), Ti(III), Ti(IV), Zr(III), Zr(IV), Hf(IV), V(III), V(IV), V(V), Nb(V), Ta(V), Fe(II), Fe(III), Co(II), Rh(II), Rh(III), Rh(IV), Cr(III), Ni(II), and Pd(II). For example, M may be Ti(IV), Zr(IV), Hf(IV), V(III), V(IV), V(V), Nb(V), and Ta(V); such as Ti(IV), Zr(IV), and Hf(IV).

The integer m has typically a value of at least 2. The number of m will depend on the number of the ligand L which is present in the self-assembled catalyst. Thus, m may be in the range of about 1 or about 2 to about 1000, for example about 1 to about 100 or about 200 or 300. However, m may also be any other integer being useful in the present invention.

X is a group which is coordinated to the transition metal atom. X may be, but is not limited to, H, F, Cl, Br, CN, N(CH₃)₂, N(CH₂CH₃)₂, CH₃, CH₂CH₃, OCH₃, OCH₂CH₃, OCH(CH₃)₃, OC(CH₃)₃, or OC₆H₆, and the like. In the case where plural X moieties are contained, X may be the same or different.

The symbol n in Formula (I) represents an integer satisfying the valence of M. The number of n depends on the valency of the transition metal M. For example, n may be an integer from about 0-5, such as about 0-4 or about 0-3. Also, n may be 1 or 2. In one embodiment n is 2 to form an octahedral metal configuration together with the two WY units of the two different ligands L. Further metal configurations may be possible depending on n.

In the above formula (I), L is a ligand which has at least two coordination units which are linked via a spacer Z so that each coordination unit can only bind to a different transition metal. This means that, for example, a ligand L having two separate coordination units can not bind the same transition metal with both coordination units. Instead of, each coordination unit binds to a different transition metal.

In the above formula (I), q may be an integer being at least 2. The number of q will depend on the number of transition metal atoms in the self-assembled catalyst. q may be in the range from about 2 to about 1000, for example about 2 to about 100. However, q may also be any other integer being useful in the present invention.

L may be a ligand according to formula (II)

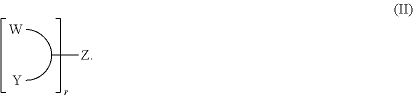

wherein

each WY unit forms a coordination unit;

r is an integer of at least 2;

Z is a bridging spacer selected from the group consisting of optionally substituted hydrocarbons having about 2 to about 100 carbon atoms and optionally substituted hetero-hydrocarbons having about 2 to about 100 carbon atoms,

wherein Z has a size, length and angle so that each coordination unit WY binds to a different transition metal;

W is a metal-coordinating moiety selected from the group consisting of an oxygen atom, a sulphur atom, a selenium atom, a nitrogen atom, and a phosphorus atom in neutral or charged form, a carbene, and an optionally substituted C₅-C₂₀ aryl;

Y is a metal-coordinating moiety selected from the group consisting of an oxygen atom, a sulphur atom, a selenium atom, a nitrogen atom, a phosphorus atom in neutral or charged form, a carbene, and an optionally substituted C₅-C₂₀ aryl;

wherein the semi-circle in the WY unit represents the hydrocarbon backbone to which the metal-coordinating moieties W and Y are bonded.

This ligand L may be prepared according to the process described below.

In the above formula (II), r may be 2, 3, 4, 5 or 6 or any integer >6. In case of r=2, the ligand L may be

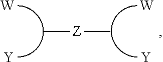

wherein in case of r=3 the ligand L may be

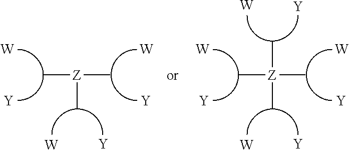

in case of r=4, and so on.

Each unit WY forms a coordination unit, i.e. one transition metal is coordinated to both W and Y of the same WY coordination unit. The semi-circle in the WY coordination unit represents the hydrocarbon backbone to which the metal-coordinating moiety W and Y are bonded. In neutral or charged form means that both W and Y may have, for example, the charge state 0 or −1 or any other charge state which contributes to a stable molecule.

The hydrocarbon backbone to which the metal-coordinating moieties W and Y are bonded may be, for example, any organic compound which is capable of linking W and Y to form the coordination unit. In one embodiment, the hydrocarbon backbone may be, but is not limited to, an optionally substituted C₆-C₂₀ aryl group, an optionally substituted C₆-C₂₀ heteroaryl group or an optionally substituted Si group. For example, W and Y may be linked to an aromatic hydrocarbon (aryl), to a Si-chain or the like.

In illustrative embodiments of the present invention the WY coordination unit may be, but is not limited to,

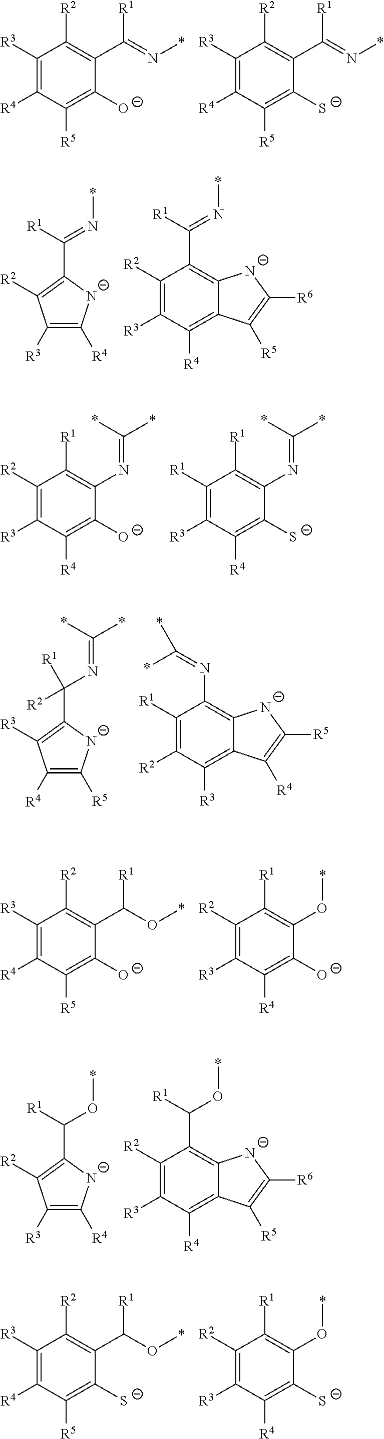

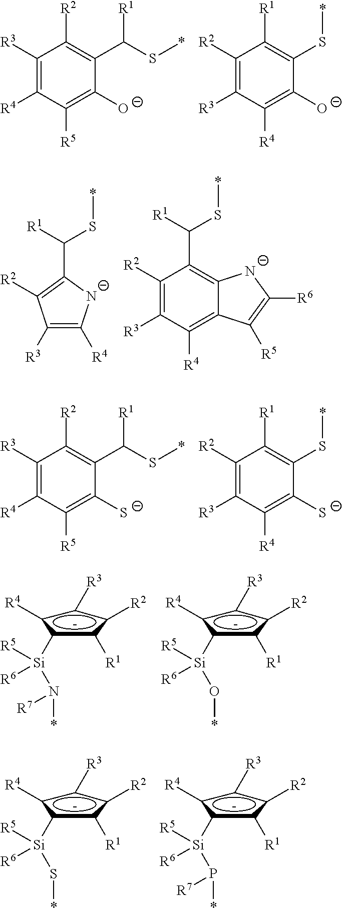

In the above formula (II), Z is a spacer molecule, wherein the term “spacer molecule” refers to an atom or group of atoms that separate two or more groups from one another by a desired number of atoms. Any group of atoms may be used to separate those groups by the desired number of atoms. In certain embodiments, spacers are optionally substituted. The spacer Z has a size, length and angle so that the at least two coordination sites WY of the ligand L can only bind to two different transition metal atoms and not to the same transition metal atom. This means, that it is not possible that every coordination site of the same ligand L may bind to one and the same transition metal, as described in the prior art.

In this respect, the term hydrocarbons having about 2 to about 100 carbon atoms refer to all possible sorts of organic compounds consisting of hydrogen and carbon, e.g. aromatic hydrocarbons (aryl), alkanes, alkenes and alkyne-based compounds, but not limited to. In one embodiment of the present invention, Z may be, but is not limited to, an optionally substituted C₃-C₂₀ alicyclic group, an optionally substituted C₆-C₂₀ aryl group, an optionally substituted C₆-C₂₀ heteroaryl group, a system of condensed nucleus fused two, three, four or five membered rings (which can optionally have heteroatoms in the ring system, such as naphthalene derivatives, anthracene derivates, quinoline, isoquinoline, quinazoline, acridinine, phenanthrene, naphthacene, chrysene, pyrene, or triphenylene, to name only a few illustrative examples)), or a system of two, three or four C₆-C₂₀ aryl groups being connected via a N-atom, a Si-atom, an C₁-C₂₀ alkyl group, an C₂-C₂₀ alkenyl group or an C₆-C₂₀ aryl group. For example, the above terms may encompass compounds such as biphenyl, terphenyl or [(R¹¹R¹²R¹³R¹⁴)C₆—(CH₂)ₖ—C₆(R¹⁵R¹⁶R¹⁷R¹⁸)], wherein k is an integer from 1 to 10, and the like. All these compounds may be optionally substituted.

The term hetero-hydrocarbons having about 2 to about 100 carbon atoms refer to all sort of organic compounds consisting of hydrogen, carbon and at least one heteroatom selected from for example N, S, O, Si or P, but mot limited to. For example, this term may encompass compounds according to the formula [(R¹¹R¹²R¹³R¹⁴)C₆—(CH₂)ₖ—C₆(R¹⁵R¹⁶R¹⁷R¹⁸)], wherein V is Si or S and v is an integer from about 1 to about 6. All these compounds may be optionally substituted.

In case of r=2 in formula (II), examples of the spacer Z include, but are not limited to, the following benzyl, pyridyl, napthtyl, biphenyl, terphenyl, anthacenyl, phenanthrenyl, or benzyl groups being connected via a N-atom, a Si-atom, or an C₁-C₂₀ alkyl group, an C₂-C₂₀ alkenyl group or an C₆-C₂₀ aryl group,

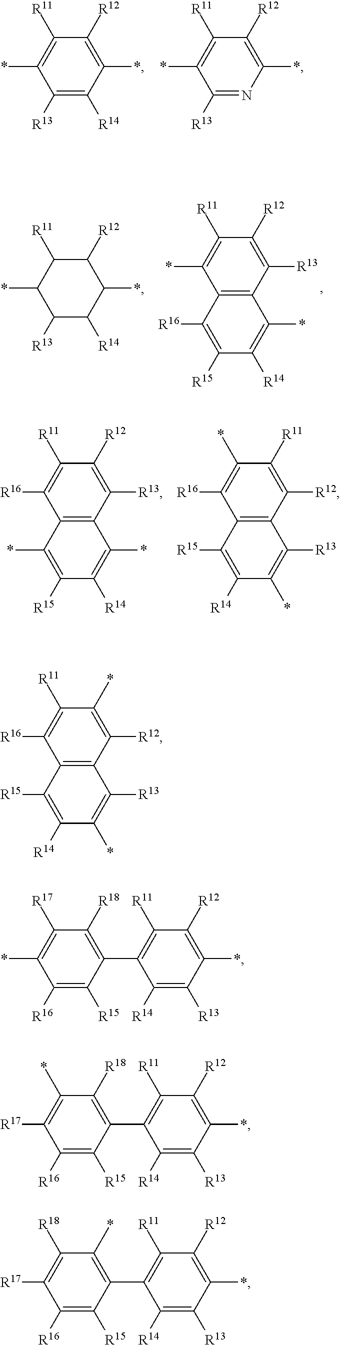

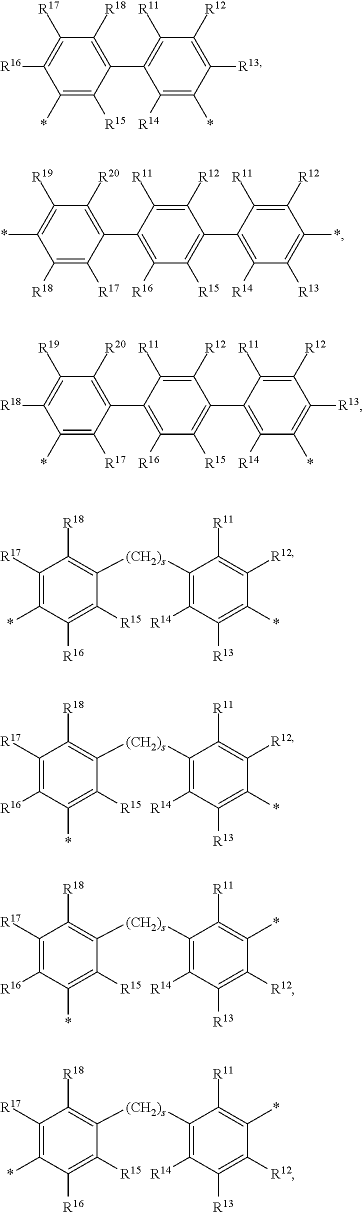

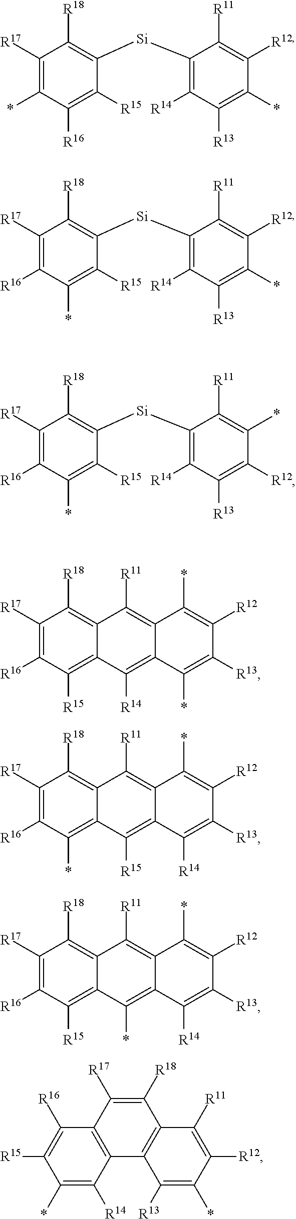

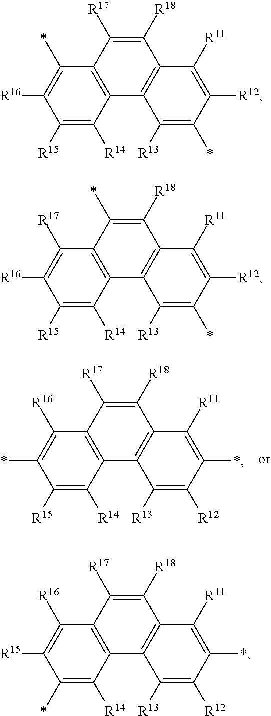

and the like. In the above formulae, s is an integer from 1 to about 20, for example from 1 to about 10. In one embodiment, s may be selected from 1, 2, 3, 4, 5 or 6.

In case of r=3 in formula (II), Z is a tri-linker. This means that three of the WY coordination units may be bonded to the same spacer. Examples of the such spacer Z may be, but are not limited to,

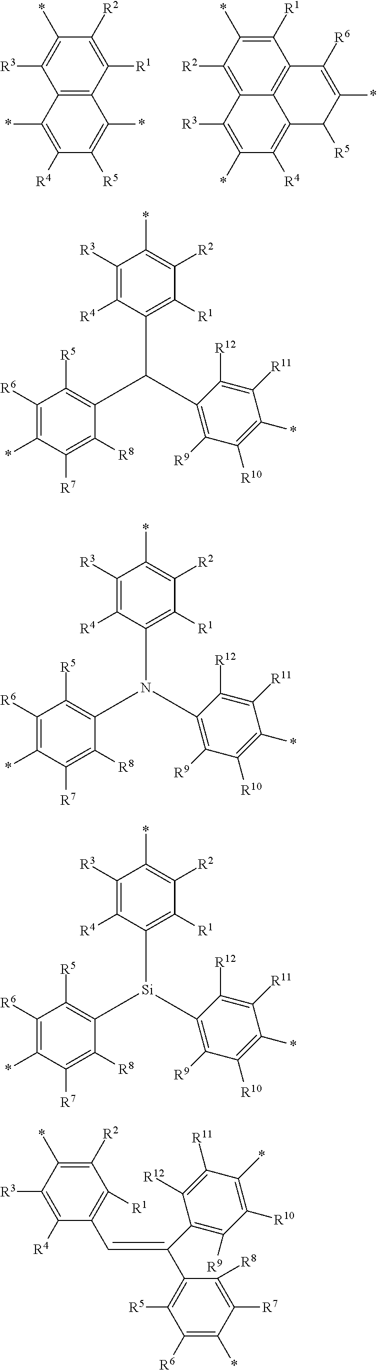

and the like.

In case of r=4 in formula (II), Z is a tetrakis-linker. This means that four of the WY coordination units may be bonded to the same spacer. Examples of such spacer Z may be, but are not limited to,

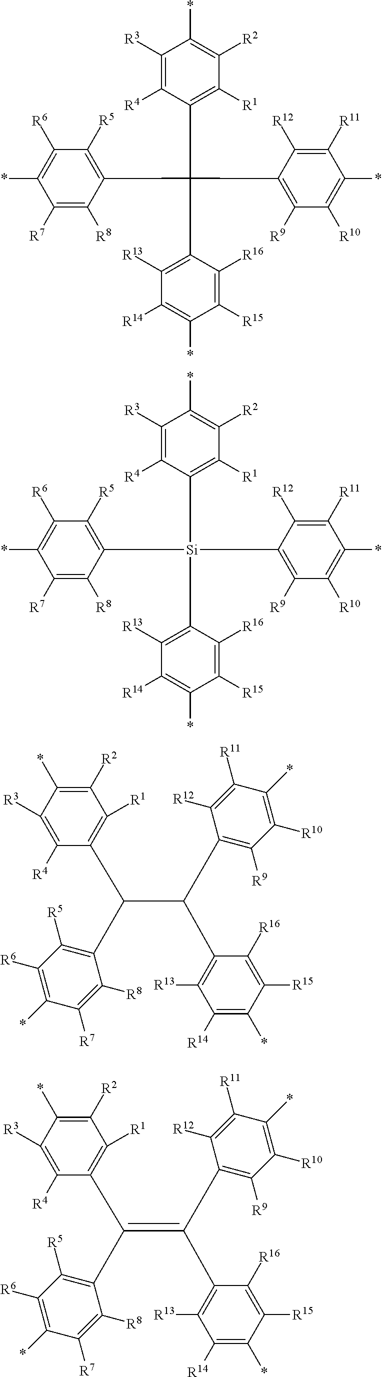

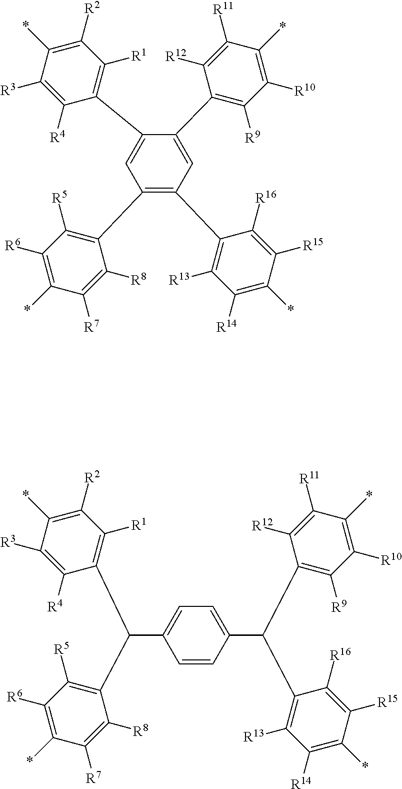

and the like.

Besides the above examples, Z may also have 5 or more than five linking sites, i.e. r in formula (II) may be 5 or 6 or even more. In addition, Z may also be a polymeric backbone having a plurality of linking sites forming a macro polymeric multi-linker. The polymeric backbone may be, for example, polyethylene, polypropylene, and the like.

R and R¹ to R²⁰ in the above or below formulas may be the same or different and are each selected from the group consisting of H, optionally substituted straight-chain or branched C₁-C₂₀ alkyl, optionally substituted straight-chain or branched C₂-C₂₀ alkenyl, optionally substituted straight-chain or branched C₂-C₂₀ alkynyl, optionally substituted C₆-C₂₀ aryl, optionally substituted C₆-C₂₀ heteroaryl, halogen, OH, NO₂, and CN, wherein two or more of R¹ to R²⁰ may be bonded to each other to form a ring.

The term “optionally substituted straight-chain or branched C₁-C₂₀ alkyl” represented by R¹ to R²⁰ refers to a fully saturated aliphatic hydrocarbon. Whenever it appears here, a numerical range, such as 1 to 20 or C₁-C₂₀ refers to each integer in the given range, e.g. it means that an alkyl group comprises only 1 carbon atom, 2 carbon atoms, 3 carbon atoms etc. up to and including 20 carbon atoms. Examples of alky groups may be, but are not limited to, methyl, ethyl, n-propyl, isopropyl, n-butyl, isobutyl, s-butyl, t-butyl, tert.-amyl.pentyl, n-hexyl, n-heptyl, n-octyl, n-nonyl or n-decyl and the like.

The term “optionally substituted straight-chain or branched C₂-C₂₀ alkenyl” refers to an aliphatic hydrocarbon having one or more carbon-carbon double bonds. Examples of alkenyl groups may be, but are not limited to, ethenyl, propenyl, allyl or 1,4-butadienyl and the like.

The term “optionally substituted straight-chain or branched C₂-C₂₀ alkynyl” refers to an aliphatic hydrocarbon having one or more carbon-carbon triple bonds. Examples of alkynyl groups may be, but are not limited to, ethynyl, propynyl, butynyl, and the like.

The term “optionally substituted C₁-C₂₀ alkoxy” refers to a group of formula —OR, wherein R is a C₁-C₂₀ alkyl group. Examples of alkoxy groups may be, but are not limited to, methoxy, ethoxy, propoxy, and the like.

The term “optionally substituted C₃-C₁₀ alicyclic group” refers to a group comprising a non-aromatic ring, wherein each of the atoms forming the ring is a carbon atom. Such rings may be formed by 3 to 10 carbon atoms. Examples of alicyclic groups may be, but are not limited to, cyclopropane, cyclobutane, cyclopentane, cyclopentene, cyclopentadiene, cyclohexane, cyclohexene, cycloheptane, cycloheptene and the like.

The term “optionally substituted C₆-C₂₀ aryl” refers to an aromatic ring, wherein each of the atoms forming the ring is a carbon atom. Aromatic in this context means a group comprising a covalently closed planar ring having a delocalized n-electron system comprising 4w+2π-electrons, wherein w is an integer of at least 1, for example 1, 2, 3 or 4. Examples of aryl groups may be, but are not limited to, phenyl, napthalenyl, phenanthrenyl, anthracenyl, tetralinyl, fluorenyl, indenyl, and indanyl, and the like.

The term “optionally substituted C₆-C₂₀ heteroaryl” refers to an aromatic heterocycle. Heteroaryls may comprise at least one or more oxygen atoms or at least one or more sulphur atoms or one to four nitrogen atoms or a combination thereof. Examples of heteroaryl groups may be, but are not limited to, furan, benzofuran, thiophene, benzothiophene, pyrrole, pyridine, indole, oxazole, benzoxazole, isoxazole, benzisoxazole, thiazole, benzothiazole, imidazole, benzimidazole, pyrazole, indazole, tetrazole, quinoline, isoquinoline, pyridazine, purine, pyrazine, furazan, triazole, benzotriazole, pteridine, phenoxazole, oxadiazole, benzopyrazole, quinolizine, cinnoline, phthalazine, quinazoline or quinoxaline, and the like.

The term “halogen” refers to fluorine, chlorine, bromine or iodine.

The term “optionally substituted Si group” refers to a group containing 1 to 5 silicon atoms which are substituted by hydrogen or an alkyl group or an aryl group. Examples of a Si group may be, but are not limited to, monosilane, methylsilyl, dimethylsily, ethylsilyl, diethylsily, phenylsily, methylphenylsilyl, and the like.

The term “a system of condensed nucleus” refers to compounds having at least two aromatic or non-aromatic condensed ring systems. Examples of condensed nucleus may be, but are not limited to, decalin, hydrindane, napthalene, anthracene, phenanthrene, naphthacene, pentacene, hexacene, pyrene, indene, fluorene, and the like.

The term “a system of two, three or four optionally substituted C₆-C₂₀ aryl groups being connected via a N-atom, a Si-atom, an C₁-C₂₀ alkyl group, an C₂-C₂₀ alkenyl group or an C₆-C₂₀ aryl group” refers to compounds having a N-atom, a Si-atom, an alkyl group, an alkenyl group or an aryl group as a central bonding unit to which two, three or four aryl groups are bonded.

Unless otherwise indicated, the term “optionally substituted,” refers to a group in which none, one, or more than one of the hydrogen atoms has been replaced with one or more group(s) independently selected from the group consisting of alkyl, aryl, heteroaryl, hydroxy, alkoxy, halogen, carbonyl, C-amido, N-amido, isocyanato, thiocyanato, isothiocyanato, nitro, silyl, and amino, including mono- and di-substituted amino groups, and the protected derivatives of amino groups. In embodiments in which two or more hydrogen atoms have been substituted, the substituent groups may be linked to form a ring.

The term “linked to form a ring” refers to the circumstance where two atoms that are bound either to a single atom or to atoms that are themselves ultimately bound, are each bound to a linking group, such that the resulting structure forms a ring. The resulting ring comprises the two atoms, the atom (or atoms) that previously linked those atoms, and the linker.

In one embodiment of the present invention the ligand L may be, but is not limited to,

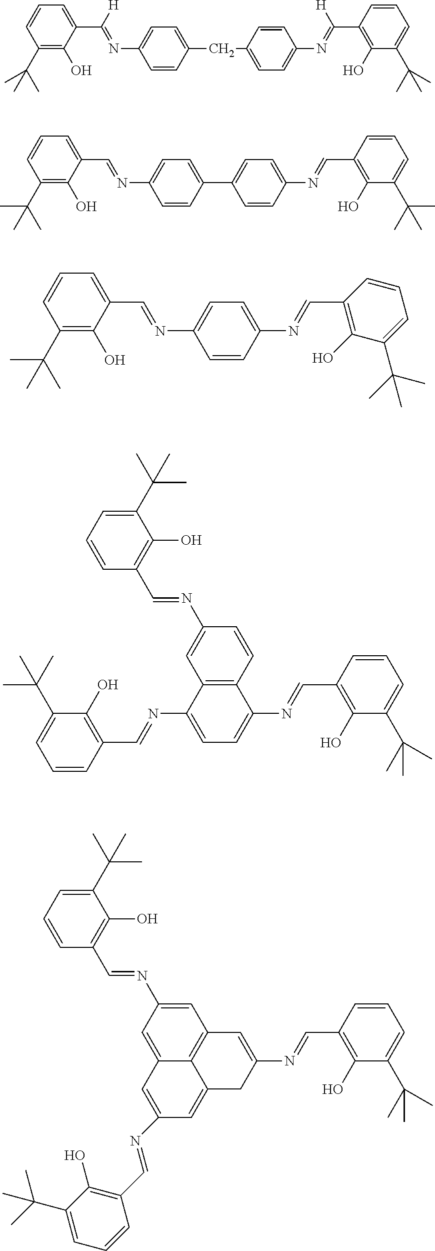

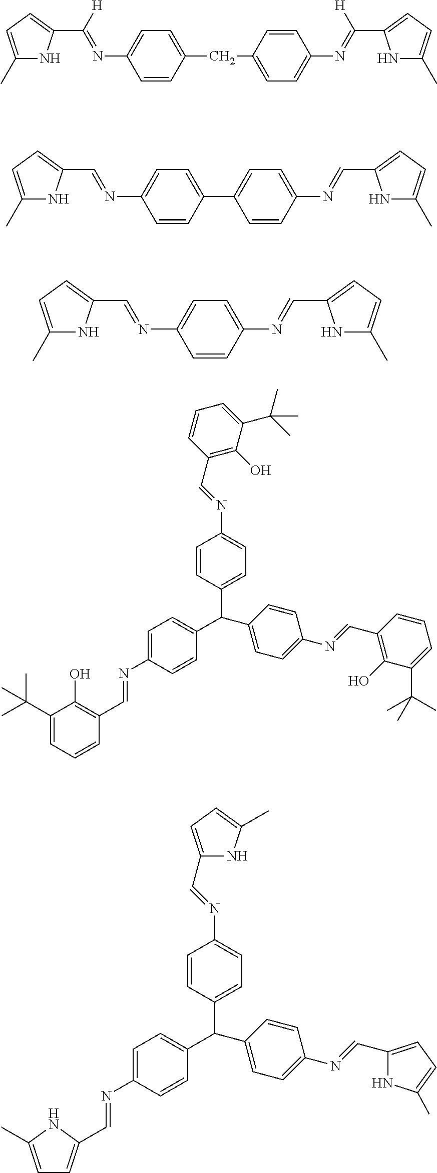

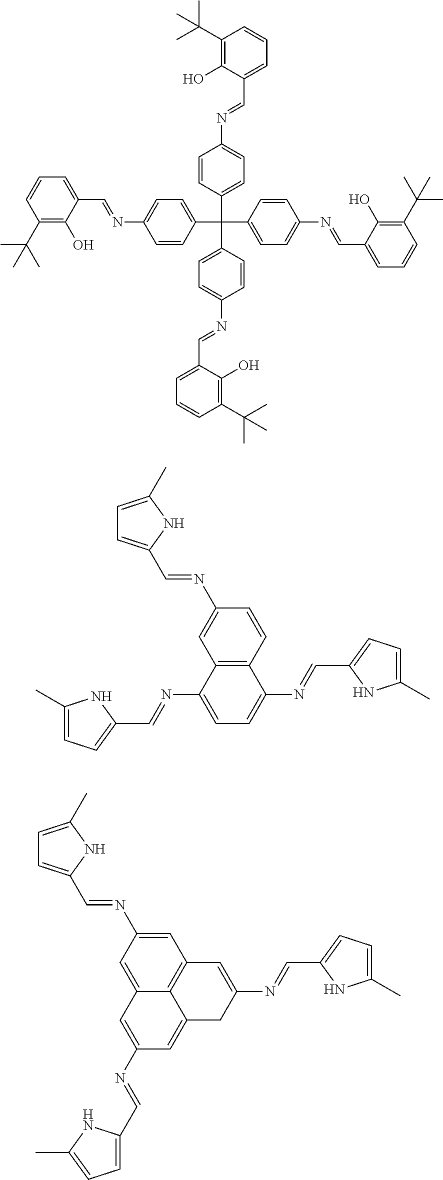

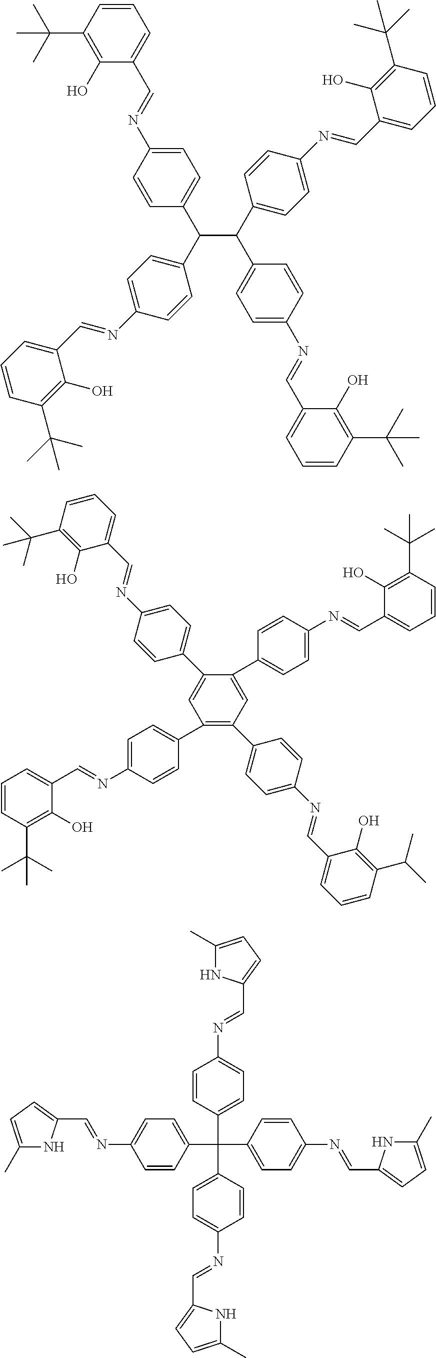

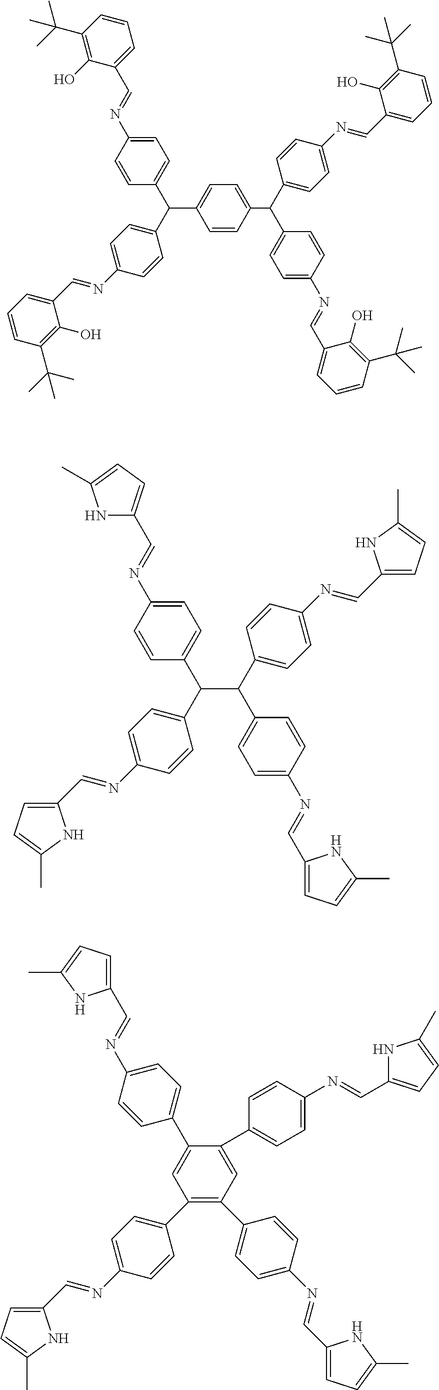

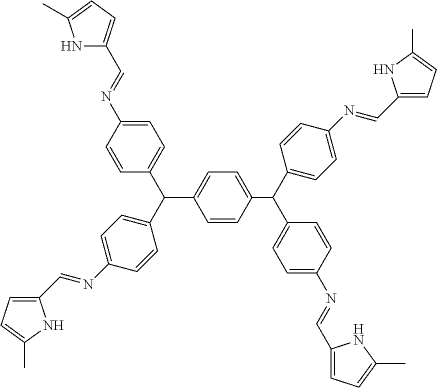

and the like.

In one embodiment the ligand L may be

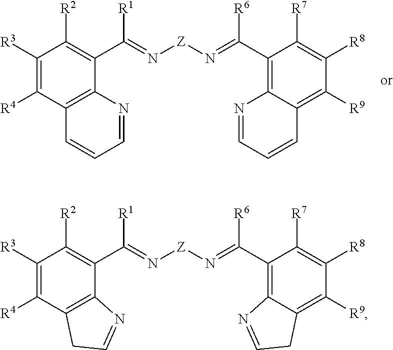

wherein Z and R¹ to R⁹ are as defined above.

The molar ration of the coordination unit WY to the transition metal may be in the range of about 0.5:1 to about 6:1, for example about 1:1 to about 3:1.

In general, the ligand compounds L may be prepared via a Schiff-Base condensation of the respective aldehyde or ketone and the amino substituted spacer molecule. Depending on the desired geometry of the ligand, the spacer molecule may have more than one amino substituent in order to react with more than one aldehyde and/or ketone. For example, the ligand may be prepared by a Schiff-Base condensation between an aldehyde or ketone with a di-aniline, tri-aniline or tetrakis-aniline. For example, the aldehyde or ketone may include, but is not limited to,

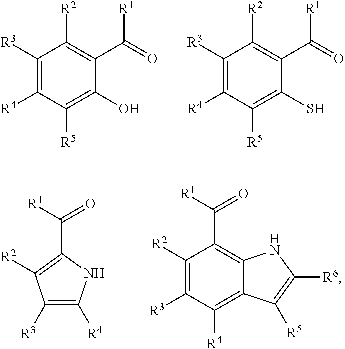

and the like, wherein R¹ to R⁶ are as described above. The di-aniline, tri-aniline or tetrakis-aniline may include, but is not limited to,

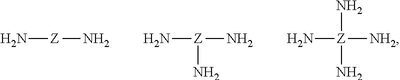

and the like wherein Z is as described above.

In an alternative embodiment, the ligand compound L may also be prepared by a Schiff-Base condensation between an aniline and an di-aldehyde/di-ketone, tri-aldehyde/tri-ketone or tetrakis-aldehyde/tetrakis-ketone. The aniline may include, but is not limited to,

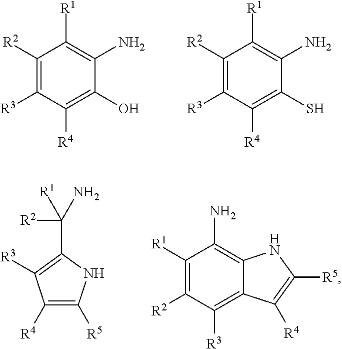

wherein R¹ to R⁵ are as described above. The di-aldehyde/di-ketone, tri-aldehyde/tri-ketone or tetrakis-aldehyde/tetrakis-ketone may include, but is not limited to,

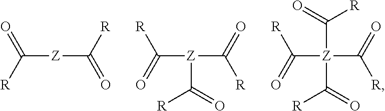

and the like wherein R and Z are as described above.

It will be understood that any other combination of aldehydes or ketones with the respective aniline compounds will be possible in the present invention to prepare the ligand compounds described above.

In the above described process for preparing the ligand compound L according to the present invention the Schiff-Base condensation may be promoted by an acid catalyst or a solid catalyst. The acid catalyst may include, but is not limited to, formic acid, acetic acid, p-toluenesulfonic acid or a Lewis acid and the like.

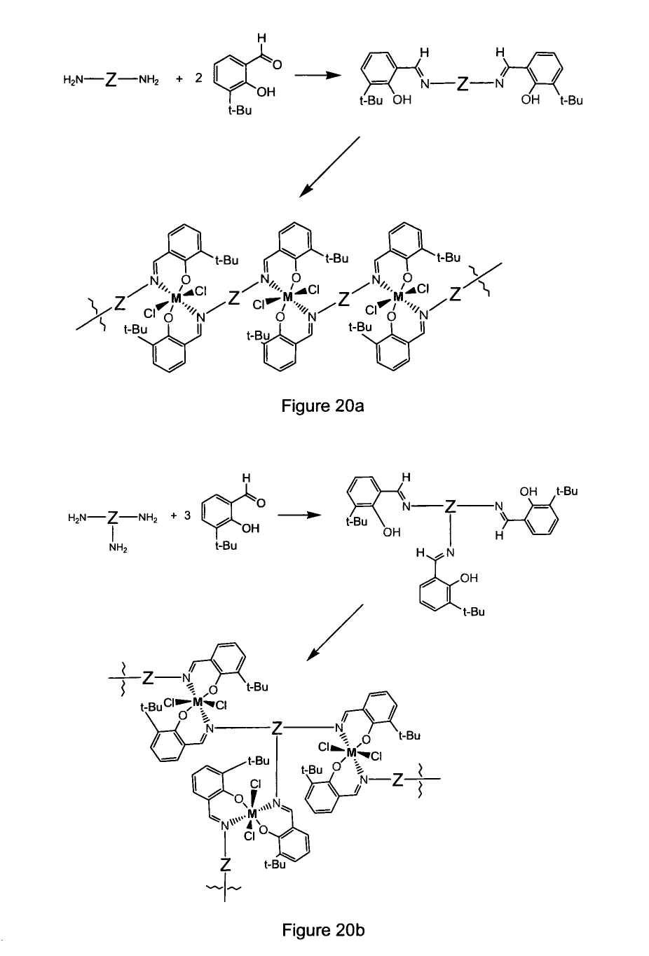

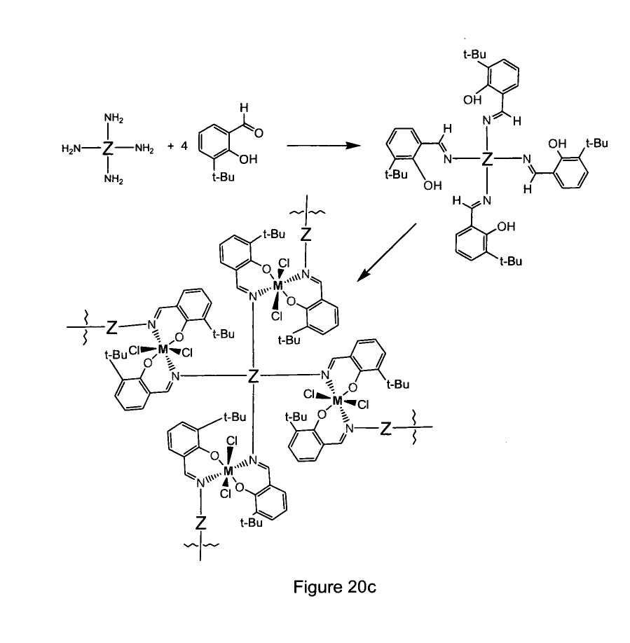

Following reaction with an organolithium compound or sodium hydride (NaH), the formed ligand compound is reacted with the respective metal compound to form the catalyst of the present invention. The general principle of this type of reaction can be seen in FIGS. 20a to 20c. In FIG. 20a the preparation of a bi-linker based ligand and a self-assembled catalyst is shown, in FIG. 20b the preparation of a tri-linker based ligand and a self-assembled catalyst is shown, and in FIG. 20c the preparation of a tetrakis-linker based ligand and a self-assembled catalyst is shown (Z is one of the spacer molecules described in the present invention).

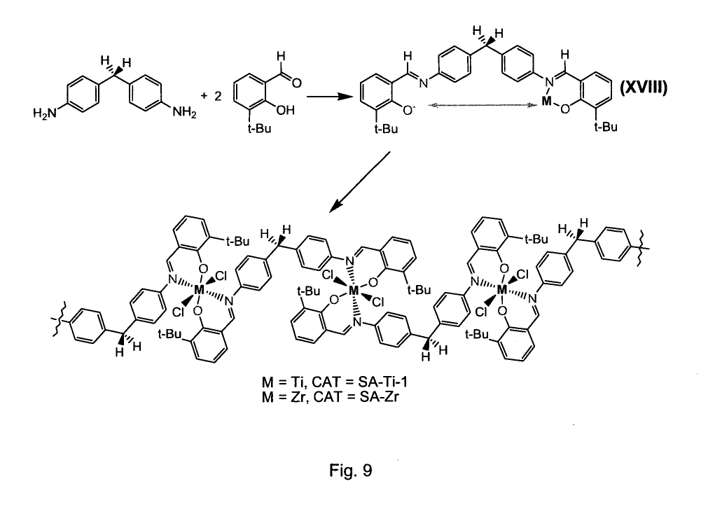

One illustrative example of this preparation method can be seen in FIG. 9, wherein the bis-phenoxy-imine ligand (XVIII) is prepared via a Schiff-Base condensation between 4,4″-diaminodiphenylmethane and 3-tert.-butyl-2-hydroxy benzaldehyde. After reaction with n-butyllithium the formed dilithium salt of bis-phenoxy-imine reacts with titanium/zirconium tetrachloride affording the self-assembled catalyst in quantitative yield.

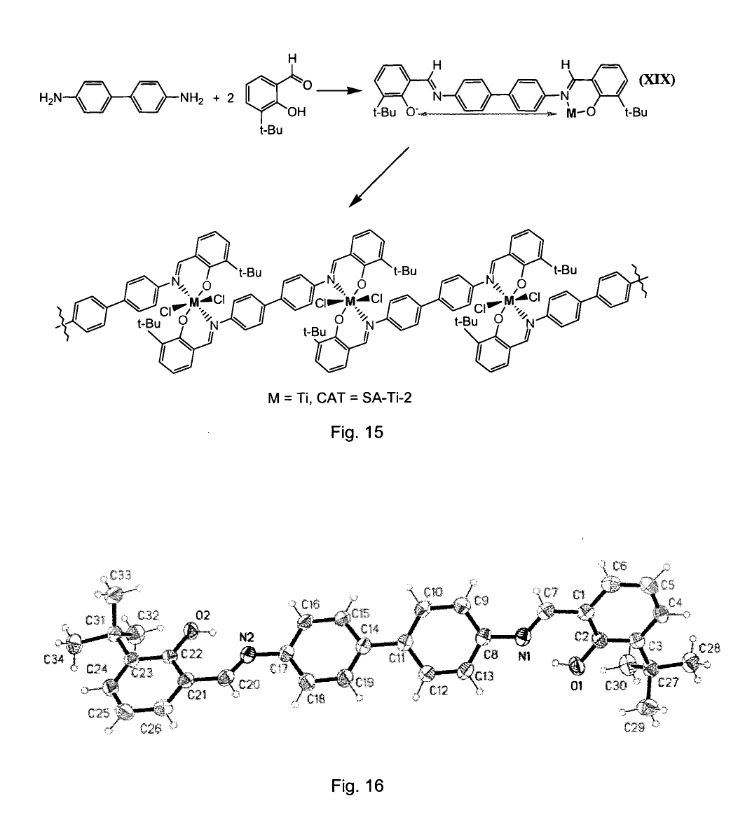

Another example of this preparation method can be taken from FIG. 15, wherein the bis-phenoxy-imine ligand (XIX) is prepared via a Schiff-Base condensation between benzidine and 3-tert.-butyl-2-hydroxy benzaldehyde. The molecular structure of bis-phenoxy-imine ligand (XIX) has been confirmed by single crystal X-Ray diffraction as shown in FIG. 16, which clearly shows that the two NO coordination units of this compound are separated by a biphenyl group. As the space between the coordination units is too big due to the biphenyl spacer, each coordination unit has to coordinate with a different metal atom to form the self-assembled catalyst structure as exemplarily shown in FIG. 15.

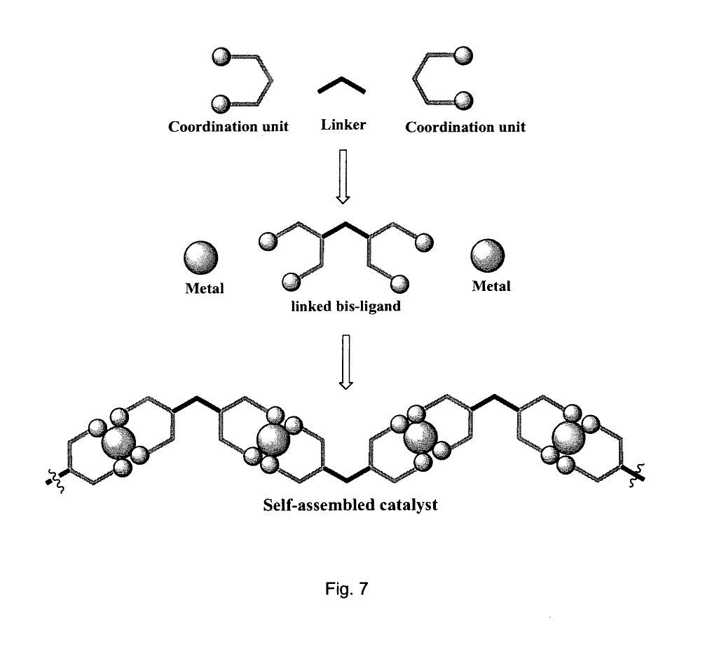

The strategy of the present invention is that the specific coordination geometry of the ligand L does not allow the at least two WY coordination units of L to coordinate with one and the same transition metal to form a mono-nuclear complex because of the spacer's size, length and angle, hence the at least two WY units have to coordinate with two or more different transition metals, thus forming self-assembled multi-nuclear catalysts. This concept can be exemplarily taken from FIG. 7, wherein the ligand L is formed by the two coordination sites and the spacer (linker). Each site of the linked bis-ligand coordinates to one metal atom such that self-assembling starts to achieve long-lived highly efficient polymerization catalyst.

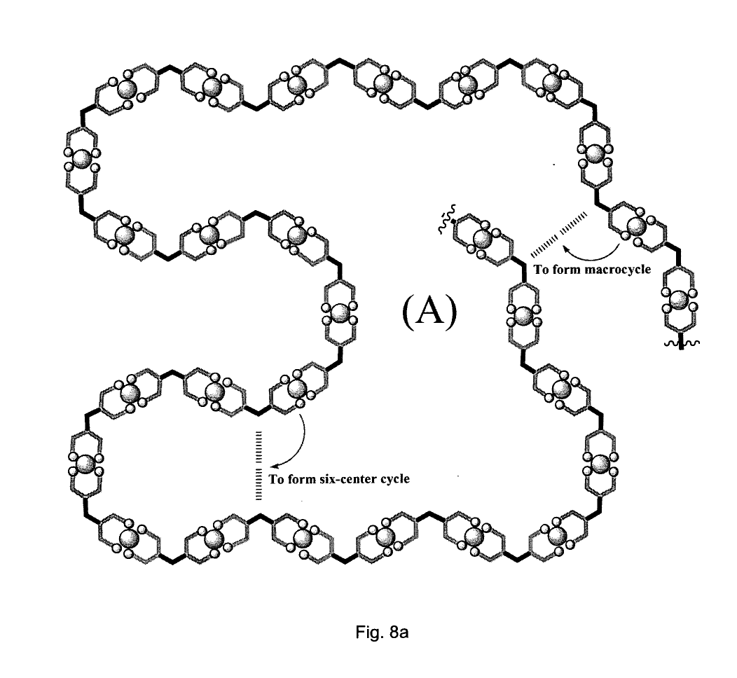

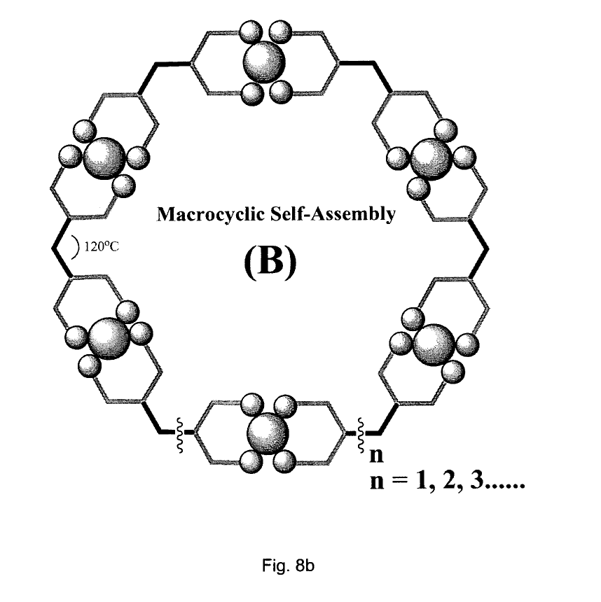

The self-assembling structure may be linear or macrocyclic as can be seen in FIGS. 8a and 8b. The kind of structure of the self-assembled catalyst will depend on the geometry of the used spacer Z and the kind and number of the substituents of the ligand L. Depending on the number of the linking sites on the spacer Z, the self-assembled catalyst of the present invention may form, for example, a 3-dimensional framework.

The self-assembled olefin polymerization catalyst of the present invention may be used together with at least one co-catalyst. In this case a catalytic system for olefin polymerization or copolymerization is formed, which may be used as such or which may be used in connection with other catalyst compounds or components necessary in the polymerization process. The at least one co-catalyst of the present invention may be, but is not limited to, an organometallic compound, an organoaluminum oxy-compound, or an ionizing ionic compound, and the like.

In one embodiment, the co-catalyst may be selected from organometallic compounds, wherein the organometallic compound may be, but is not limited to, an organometallic compound of metals of Group 1, Group 2, Group 12 and Group 13 of the Periodic Table. For example, in case of Al compounds, the compounds may be represented by the general Formula:

R^{a}ₘAl(OR^{b})ₙHₚX_{q}

wherein R^{a} and R^{b}, which may be the same or different, may be a hydrocarbon group of 1 to 15, for example 1 to 4 carbon atoms; X may be a halogen atom; and m, n, p and q are integers satisfying the conditions of 0<m≦3, 0≦n<3, 0≦p<3, 0≦q<3 and m+n+p+q=3.

Examples of the above organoaluminum compound may include the following compounds, but are not limited to, organoaluminum compounds represented by the general formula

R^{a}ₘAl(OR^{b})₃₋ₙ,

wherein R^{a} and R^{b}, which may be the same or different, may be a hydrocarbon group of 1 to 15, for example 1 to 4 carbon atoms; and m may be a number satisfying the condition of 1.5≦m≦3.

Further exemplary compounds are represented by the general formula

R^{a}ₘAlX₃₋ₘ

wherein R^{a} is a hydrocarbon group of 1 to 15, for example 1 to 4 carbon atoms; X is a halogen atom; and m may be an integer satisfying the condition of 0<m<3.

Further exemplary compounds are represented by the general formula

R^{a}ₘAlH₃₋ₘ

wherein R^{a} is a hydrocarbon group of 1 to 15, for example 1 to 4 carbon atoms; and m may be an integer satisfying the condition of 2≦m<3.

Further exemplary compounds are represented by the general formula

R^{a}ₘAl(OR^{b})ₙX_{q}

wherein R^{a} and R^{b}, which may be the same or different, may be a hydrocarbon group of 1 to 15, for example 1 to 4 carbon atoms; X may be a halogen atom; and m, n and q are integers satisfying the conditions of 0<m≦3, 0<n≦3, 0≦q<3 and m+n+q=3.

Specific examples of the above organoaluminum compounds may include, but are not limited to, tri-n-alkylaluminums, such as trimethylaluminum, triethylaluminum, tri-n-butylaluminum, tripropylaluminum, tripentylaluminum, trihexylaluminum, trioctylaluminum and tridecylaluminum; branched-chain trialkylaluminums, such as triisopropylaluminum, triisobutylaluminum, tri-sec-butylaluminum, tri-t-butylaluminum, tri-2-methylbutylaluminum, tri-3-methylbutylaluminum, tri-2-methylpentylaluminum, tri-3-methylpentylaluminum, tri-4-methylpentylaluminum, tri-2-methylhexylaluminum, tri-3-methylhexylaluminum and tri-2-ethylhexylaluminum; tricycloalkylaluminums, such as tricyclohexylaluminum and tricyclooctylaluminum; triarylaluminums, such as triphenylaluminum and tritolylaluminum; dialkylaluminum hydrides, such as diisobutylaluminum hydride; trialkenylaluminums represented by the formula (i-C₄H₉)ₓAl_{y}(C₅H₁₀)_{z} (wherein x, y and z are positive numbers, and z≧2x), such as triisoprenylaluminum; alkylaluminum alkoxides, such as isobutylaluminum methoxide, isobutylaluminum ethoxide and isobutylaluminum isopropoxide; dialkylaluminum alkoxides, such as dimethylaluminum methoxide, diethylaluminum ethoxide and dibutylaluminum butoxide; alkylaluminum sesquialkoxides, such as ethylaluminum sesquiethoxide and butylaluminum sesquibutoxide; partially alkoxylated alkylaluminums having an average composition, represented by R^{a}_{2.5}Al(OR^{b})_{0.5}; dialkylaluminum aryloxides, such as diethylaluminum phenoxide, diethylaluminum(2,6-di-t-butyl-4-methylphenoxide), ethylaluminum bis-(2,6-di-t-butyl-4-methylphenoxide), diisobutylalumium(2,6-di-t-butyl-4-methylphenoxide) and isobutylaluminum bis(2,6-di-t-butyl-4-methylphenoxide); dialkylaluminum halides, such as dimethylaluminum chloride, diethylaluminum chloride, dibutylaluminum chloride, diethylaluminum bromide and diisobutylaluminum chloride; alkylaluminum sesquihalides, such as ethylaluminum sesquichloride, butylaluminum sesquichloride and ethylaluminum sesquibromide, partially halogenated alkylaluminums, such as alkylaluminum dihalides, e.g., ethylaluminum dichloride, propylaluminum dichloride and butylaluminum dibromide; dialkylaluminum hydrides, such as diethylaluminum hydride and dibutylaluminum hydride; partially hydrogenated alkylaluminums, such as alkylaluminum dihydrides, e.g., ethylaluminum dihydride and propylaluminum dihydride; and partially alkoxylated and halogenated alkylaluminums, such as ethylaluminum ethoxychloride, butylaluminum butoxychloride and ethylaluminum ethoxybromide.

Also employable are compounds analogous to the above organoaluminum compounds. For example, there can be mentioned organoaluminum compounds wherein two or more aluminum compounds are combined through a nitrogen atom, such as (C₂H₅)₂AlN(C₂H₅)Al(C₂H₅)₂.

In one embodiment, the above organometallic compound may be a compound of a Group 1 metal of the Periodic Table and aluminum represented by the general formula

M²AlR^{a}₄

wherein M² is Li, Na or K; and R^{a} is a hydrocarbon group of 1 to 15, for example 1 to 4 carbon atoms. Examples of these organoaluminum compounds include, but are not limited to, LiAl(C₂H₅)₄ and LiAl(C₇H₁₅)₄, and the like.

In a further embodiment, the above organometallic compound may be a compound of a Group 2 Metal or a Group 12 Metal of the Periodic Table represented by the general Formula

R^{a}R^{b}M³

wherein R^{a} and R^{b}, which may be the same or different, may be a hydrocarbon group of 1 to 15, preferably 1 to 4 carbon atoms; and M³ is Mg, Zn or Cd.

Further, other compounds such as methyllithium, ethyllithium, propyllithium, butyllithium, methylmagnesium bromide, methylmagnesium chloride, ethylmagnesium bromide, ethylmagnesium chloride, propylmagnesium bromide, propylmagnesium chloride, butylmagnesium bromide, butylmagnesium chloride, dimethylmagnesium, diethylmagnesium, dibutylmagnesium and butylethylmagnesium may also be employable as the above organometallic compound. Furthermore, combinations of compounds capable of forming the aforesaid organoaluminum compounds in the polymerization system, e.g., a combination of halogenated aluminum and alkyllithium and a combination of halogenated aluminum and alkylmagnesium, are also employable. The above organometallic compounds may be used singly or in combination.

The organoaluminum oxy-compound may be conventional aluminoxane or a benzene-insoluble organoaluminum oxy-compound as exemplified in JP-A-2 (1990)/78687. The conventional aluminoxane can be prepared by, for example, the following processes, and is usually obtained as a hydrocarbon solvent solution:

(1) A process wherein such an organoaluminum compound as trialkylaluminum is added to a hydrocarbon medium suspension of a compound containing absorbed water or a salt containing water of crystallization, such as magnesium chloride hydrate, copper sulfate hydrate, aluminum sulfate hydrate, nickel sulfate hydrate or cerous chloride hydrate, to react the absorbed water or the water of crystallization with the organoaluminum compound.
(2) A process wherein water, ice or water vapor is allowed to act directly on such an organoaluminum compound as trialkylaluminum in a medium, such as benzene, toluene, ethyl ether or tetrahydrofuran.
(3) A process wherein an organotin oxide, such as dimethyltin-oxide or dibutyltin oxide, is allowed to react with such an organoaluminum compound as trialkylaluminum in a medium, such as decane, benzene or toluene.

The aluminoxane may contain a small amount of an organometallic component. The solvent or the unreacted organoaluminum compound is distilled off from the recovered solution of aluminoxane and the remainder may be redissolved in a solvent or suspended in a poor solvent of aluminoxane. Examples of the organoaluminum compound used for preparing the aluminoxane include the same organoaluminum compounds as described above. The organoaluminum compounds can be used singly or in combination.

Examples of the solvent used in preparing the aluminoxane include aromatic hydrocarbons, such as benzene, toluene, xylene, cumene and cymene; aliphatic hydrocarbons, such as pentane, hexane, heptane, octane, decane, dodecane, hexadecane and octadecane; alicyclic hydrocarbons, such as cyclopentane, cyclohexane, cyclooctane and methylcyclopentane; petroleum fractions, such as gasoline, kerosine and gas oil; and halides of these aromatic, aliphatic and alicyclic hydrocarbons, particularly chlorides and bromides thereof. Also employable are ethers such as ethyl ether and tetrahydrofuran. Of the solvents, particularly preferable are aromatic hydrocarbons and aliphatic hydrocarbons.

The benzene-insoluble organoaluminum oxy-compound used in the invention preferably has a content of Al component which is soluble in benzene at about 60° C. of usually not more than about 10%, for example not more than about 5%, such as not more than about 2%, in terms of Al atom. That is, the benzene-insoluble organoaluminum oxy-compound is preferably insoluble or hardly soluble in benzene.

The organoaluminum oxy-compound employable in the invention is, for example, an organoaluminum oxy-compound containing boron, which is represented by the following formula (XX)

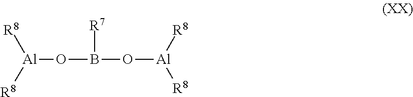

wherein R⁷ is a hydrocarbon group of 1 to 10 carbon atoms; and the groups R⁸, which may be the same or different, may be a hydrogen atom, a halogen atom or a hydrocarbon group of 1 to 10 carbon atoms.

The organoaluminum oxy-compound containing boron that is represented by the formula (XX) can be prepared by reacting an alkylboronic acid represented by the following formula (XXI) with an organoaluminum compound in an inert solvent under an inert gas atmosphere at a temperature of about −80° C. to room temperature for about 1 minute to about 24 hours:

R⁷—B—(OH)₂ (XXI)

wherein R⁷ is the same as mentioned above. Examples of the alkylboronic acid represented by the formula (XXI) include methylboronic acid, ethylboronic acid, isopropylboronic acid, n-propylboronic acid, n-butylboronic acid, isobutylboronic acid, n-hexylboronic acid, cyclohexylboronic acid, phenylboronic acid, 3,5-difluoroboronic acid, pentafluorophenylboronic acid and 3,5-bis (trifluoromethyl)phenylboronic acid. Of these, preferable are methylboronic acid, n-butylboronic acid, isobutylboronic acid, 3,5-difluorophenylboronic acid and pentafluorophenylboronic acid. These alkylboronic acids are used singly or in combination. Examples of the organoaluminum compound to be reacted with the alkylboronic acid include the same organoaluminum compounds as described for the organoaluminum compounds above. These organoaluminum compounds can be used singly or in combination.

In one embodiment the co-catalyst may be selected from organoaluminium compounds, wherein the organo aluminium compound may be, but is not limited to, trialkylaluminum such as trimethylaluminum, triethylaluminum, triisobutylaluminum, trihexylaluminum, trioctylaluminum, and tridecylaluminum; alkylaluminum halides such as diethylaluminum monochloride, diisobutylaluminum monochloride, ethylaluminum sesquichloride, and ethylaluminum dichloride; alkylaluminum hydrides such as diethylaluminum hydride, and diisobutylaluminum hydride. In one embodiment of the present invention the co-catalyst may be a methyl aluminoxane (MAO) and/or a modified methyl aluminoxane (MMAO).

The organoaluminum oxy-compounds mentioned above are used singly or in combination.

The compound that reacts with the transition metal compound to form an ion pair (also referred to as ionizing ionic compound may include, but is not limited to, Lewis acids, ionic compounds, borane compounds and carborane compounds as described in JP-A-1(1989)/501950, JP-A-1(1989)/502036, JP-A-3 (1991)/179005, JP-A-3 (1991)/179006, JP-A-3 (1991)/207703 and JP-A-3 (1991)/207704, and U.S. Pat. No. 5,321,106. Examples further include heteropoly compounds and isopoly compounds.

Examples of the Lewis acids include compounds represented by BR₃ (wherein R is a phenyl group which may have a substituent group such as fluorine, methyl or trifluoromethyl, or a fluorine atom), such as trifluoroboron, triphenylboron, tris(4-fluorophenyl)boron, tris(3,5-difluorophenyl)boron, tris(4-fluoromethylphenyl)boron, tris(pentafluorophenyl)boron, tris(p-tolyl)boron, tris(o-tolyl)boron and tris(3,5-dimethylphenyl)boron, but are not limited to.

Examples of the ionic compounds include compounds represented by the following formula (XXII)

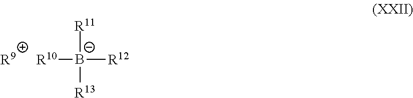

In the above formula, R⁹ may be H⁺, carbonium cation, oxonium cation, ammonium cation, phosphonium cation, cycloheptyltrienyl cation, ferrocenium cation having a transition metal, or the like. R¹⁰ to R¹³, which may be the same or different, are each an organic group, preferably an aryl group or a substituted aryl group. Examples of the carbonium cation include tri-substituted carbonium cations, such as triphenylcarbonium cation, tri(methylphenyl)carbonium cation and tri(dimethylphenyl)carbonium cation. Examples of the ammonium cation include trialkylammonium cations, such as trimethylammonium cation, triethylammonium cation, tripropylammonium cation, tributylammonium cation and tri(n-butyl)ammonium cation; N,N-dialkylanilinium cations, such as N,N-dimethylanilinium cation, N,N-diethylanilinium cation and N,N-2,4,6-pentamethylanilinium cation; and dialkylammonium cations, such as di(isopropyl)ammonium cation and dicyclohexylammonium cation. Examples of the phosphonium cation include triarylphosphonium cations, such as triphenylphosphonium cation, tri(methylphenyl)phosphonium cation and tri(dimethylphenyl)phosphonium cation.

R⁹ is preferably carbonium cation or ammonium cation, particularly preferably triphenylcarbonium cation, N,N-dimethylanilinium cation or N,N-diethylanilinium cation.

Examples of the ionic compounds further include trialkyl-substituted ammonium salts, N,N-dialkylanilinium salts, dialkylammonium salts and triarylphosphonium salts. Examples of the trialkyl-substituted ammonium salts include triethylammoniumtetra(phenyl)boron, tripropylammoniumtetra(phenyl)boron, tri(n-butyl)ammoniumtetra(phenyl)boron, trimethylammoniumtetra(p-tolyl)boron, trimethylammoniumtetra(o-tolyl)boron, tri(n-butyl)ammoniumtetra(pentafluorophenyl)boron, tripropylammoniumtetra-(o,p-dimethylphenyl)boron, tri(n-butyl)ammoniumtetra(m,m-dimethylphenyl)boron, tri(n-butyl)ammoniumtetra(p-trifluoromethylphenyl)boron, tri(n-butyl)ammoniumtetra(3,5-ditrifluoromethylphenyl) boron and tri(n-butyl)ammoniumtetra(o-tolyl)boron.

Examples of the N,N-dialkylanilinium salts include N,N-dimethylaniliniumtetra(phenyl)boron, N,N-diethylaniliniumtetra(phenyl)boron and N,N-2,4,6-pentamethylaniliniumtetra(phenyl)boron.

Examples of the dialkylammonium salts include di(1-propyl)ammoniumtetra(pentafluorophenyl)boron and dicyclohexylammoniumtetra(phenyl)boron. Examples of the ionic compounds further include triphenylcarbeniumtetrakis(pentafluorophenyl)borate, N,N-dimethylaniliniumtetrakis(pentafluorophenyl)borate, ferroceniumtetra(pentafluorophenyl)borate, triphenylcarbeniumpentaphenylcyclopentadienyl complex, N,N-diethylaniliniumpentaphenylcyclopentadienyl complex and boron compounds represented by the following formula (XXIII) or (XXIV)

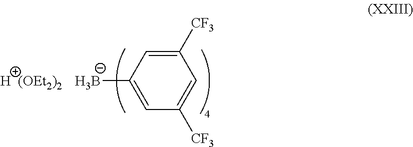

wherein Et is an ethyl group,

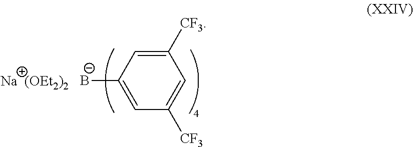

Examples of the borane compounds include, but are not limited to, decaborane; salts of anions, such as bis[tri(n-butyl)ammonium]nonaborate, bis[tri(n-butyl)ammonium]decaborate, bis[tri(n-butyl)ammonium]undecaborate, bis[tri(n-butyl)ammonium]dodecaborate, bis[tri(n-butyl)ammonium]decachlorodecaborate and bis[tri(n-butyl)ammonium]dodecachlorododecaborate; and salts of metallic borane anions, such as tri(n-butyl)ammoniumbis(dodecahydridododecaborato) cobaltate(III) and bis[tri(n-butyl)ammonium]bis(dodecahydridododecaborato) nickelate(III).

Examples of the carborane compounds may include, but are not limited to, salts of anions, such as 4-carbanonaborane, 1,3-dicarbanonaborane, 6,9-dicarbadecaborane, dodecahydrido-1-phenyl-1,3-dicarbanonaborane, dodecahydrido-1-methyl-1,3-d icarbanonaborane, undecahydrido-1,3-dimethyl-1,3-dicarbanonaborane, 7,8-dicarbaundecaborane, 2,7-d icarbaundecaborane, undecahydrido-7,8-dimethyl-7,8-dicarbaundecaborane, dodecahydrido-11-methyl-2,7-dicarbaundecaborane, tri(n-butyl)ammonium-1-carbadecaborate, tri(n-butyl)ammonium-1-carbaundecaborate, tri(n-butyl)ammonium-1-carbadodecaborate, tri(n-butyl)ammonium-1-trimethylsilyl-1-carbadecaborate, tri(n-butyl)ammoniumbromo-1-carbadodecaborate, tri(n-butyl)ammonium-6-carbadecaborate, tri(n-butyl)ammonium-6-carbadecaborate, tri(n-butyl)ammonium-7-carbaundecaborate, tri(n-butyl)ammonium-7,8-dicarbaundecaborate, tri(n-butyl)ammonium-2,9-dicarbaundecaborate, tri(n-butyl)ammoniumdodecahydrido-8-methyl-7,9-dicarbaundecaborate, tri(n-butyl)ammoniumundecahydrido-8-ethyl-7,9-dicarbaundecaborate, tri(n-butyl)ammoniumundecahydrido-8-butyl-7,9-dicarbaundecaborate, tri(n-butyl)ammoniumundecahydrido-8-allyl-7,9-dicarbaundecaborate, tri(n-butyl)ammoniumundecahydrido-9-trimethylsilyl-7,8-dicarbaundecaborate and tri(n-butyl)ammoniumundecahydrido-4,6-dibromo-7-carbaundecaborate; and salts of metallic carborane anions, such as tri(n-butyl)ammoniumbis(nonahydrido-1,3-dicarbanonaborato) cobaltate(III), tri(n-butyl)ammoniumbis(undecahydrido-7,8-dicarbaundecaborato)ferrate(III), tri(n-butyl)ammoniumbis(undecahydrido-7,8-dicarbaundecaborato)cobaltate(III), tri(n-butyl)ammoniumbis(undecahydrido-7,8-dicarbaundecaborato)nickelate(III), tri(n-butyl)ammoniumbis(undecahydrido-7,8-dicarbaundecaborato)cuprate(III), tri(n-butyl)ammoniumbis(undecahydrido-7,8-dicarbaundecaborato)aurate(III), tri(n-butyl)ammoniumbis(nonahydrido-7,8-dimethyl-7,8-dicarbaundecaborato)ferrate(III), tri(n-butyl)ammoniumbis(nonahydrido-7,8-dimethyl-7,8-dicarbaundecaborato)chromate(III), tri(n-butyl)ammoniumbis(tribromooctahydrido-7,8-dicarbaundecaborato)cobaltate(III), tris[tri(n-butyl)ammonium]bis(undecahydrido-7-carbaundecaborato)chromate(III), bis[tri(n-butyl)ammonium]bis(undecahydrido-7-carbaundecaborato)manganate(IV), bis[tri(n-butyl)ammonium]bis(undecahydrido-7-carbaundecaborato)cobaltate(III) and bis[tri(n-butyl)ammonium]bis(undecahydrido-7-carbaundecaborato)nickelate(IV).

The heteropoly compounds comprise an atom selected from silicon, phosphorus, titanium, germanium, arsenic and tin, and at least one atom selected from vanadium, niobium, molybdenum and tungsten. Examples of the heteropoly compounds include without limiting thereto phosphovanadic acid, germanovanadic acid, arsenovanadic acid, phosphoniobic acid, germanoniobic acid, siliconomolybdic acid, phosphomolybdic acid, titanomolybdic acid, germanomolybdic acid, arsenomolybdic acid, stannnomolybdic acid, phosphotungstic acid, germanotungstic acid, stannotungstic acid, phosphomolybdovanadic acid, phosphotungstovanadic acid, germanotungstovanadic acid, phosphomolybdotungstovanadic acid, germanomolybdotungstovanadic acid, phosphomolybdotungstic acid and phosphomolybdoniobic acid, salts of these acids with a metal of Group 1 or Group 2 of the Periodic Table, such as lithium, sodium, potassium, rubidium, cesium, beryllium, magnesium, calcium, strontium or barium, and organic salts of these acids with a triphenylethyl salt.

In one embodiment of the present invention, the co-catalyst may be a conventional methyl aluminoxane (MAO), a modified methyl aluminoxane (MMAO), a metal salt of (C₆F₅)₄B⁻ or a combination of an alkyl aluminium compound with MgCl₂.

The ionizing ionic compounds mentioned above can be used singly or in combination.

The catalyst:co-catalyst ratio may be about in the range of about 1:1 to about 1:5000, for example in the range of about 1:10 to about 1:2000.

The self-assembled olefin polymerization catalyst of the present invention may be supported by an inorganic or organic carrier material. The inorganic compound for the carrier may include, but is not limited to, inorganic oxides, inorganic chlorides, and other inorganic salts such as sulfates, carbonates, phosphates, nitrates, silicates, and the like.

In one embodiment the inorganic compounds for the carrier may be inorganic oxides such as silica, titania, alumina, zirconia, chromia, magnesia, boron oxide, calcium oxide, zinc oxide, barium oxide, silica xerogel, silica aerogel, and mixtures thereof such as silica/chromia, silica/chromia/titania, silica/alumina, silica/titania, silica/magnesia, silica/magnesia/titania, aluminum phosphate gel. The inorganic oxide may contain a carbonate salt, a nitrate salt, a sulphate salt, an oxide, including Na₂CO₃, K₂CO₃, CaCO₃, MgCO₃, Na₂SO₄, Al₂(SO₄)₃, BaSO₄, KNO₃, Mg(NO₃)₂, Al(NO₃)₃, Na₂O, K₂O, and Li₂O.

The inorganic compound used in the present invention may also include, but is not limited to, inorganic compound polymers such as carbosiloxane, phosphazyne, siloxane, and polymer/silica composites.

In one embodiment of the present invention the inorganic carrier material may be, but is not limited to, silica, alumina, titania, magnesium chloride, and mixtures thereof.

In a further embodiment of the present invention, the organic compound useful as the carrier may include, but is not limited to, polyethylene, ethylene/[α]-olefin copolymers, polypropylene, polystyrenes, functionalized polyethylenes, functionalized polypropylenes, functionalized polystyrenes, polyketones and polyesters.

Another embodiment of the present invention is directed to a process for polymerization or copolymerization of an olefin or a mixture of olefins in the presence of the self-assembled olefin polymerization catalyst according to the invention and optionally in the presence of at least one of the above mentioned co-catalysts.

The temperature of polymerization with the olefin polymerization catalyst is in the range usually from about −50 to about +200° C., such as from about −20° C. to about 150° C. In another embodiment, the temperature is in the range of about 0° C. to about 100° C. In another embodiment, the temperature may be in the range of about 40 to about 60° C. The polymerization pressure is in the range usually from atmospheric pressure (about 0.1 MPa) to about 10 MPa. For example, the pressure may be in the range of about 0.5 to about 1.0 MPa. The polymerization may be conducted by any of a batch system, a semicontinuous system, and a continuous system or the like. The polymerization can be conducted in two or more steps under different reaction conditions.

The molecular weight of the produced olefin polymer may be controlled, for example, by presence of hydrogen in the polymerization system or the change of polymerization temperature or pressure. With the catalyst of the present invention polymers having a number molecular weight from about 3.000 to about 3.000.000 can be obtained. It is very useful that the catalysts of the present invention can produce low molecular weight polyolefins as well as ultra high molecular weight polyolefins of more than one million with narrow molecular weight distribution.

The molecular weight may depend on several factors. For example, the substituents of the catalyst system may influence the molecular weight, for example bulkier substituents (in particular adjacent to the WY coordination unit) may give higher molecular weight. Further, a higher ethylene pressure may also contribute to a higher molecular weight. Also, a higher hydrogen pressure may lead to a lower molecular weight. The kind of metal atom in the catalyst plays also a decisive role. For example, the use of titanium may give higher molecular weight than the use of zirconium. The present invention also revealed that self-assembling increased molecular weight compared to corresponding mono-nuclear catalyst. In general it may be stated without being bound to any particular theory that higher molecular weight may give a higher melting point and better mechanical properties.

The olefins which can be polymerized according to the present invention include linear or branched α-olefins of 2-30, for example 2-20 carbon atoms. In one embodiment the olefins may be, but are not limited to, ethylene, propylene, 1-butene, 2-butene, 1-pentene, 3-methyl-1-butene, 1-hexene, 4-methyl-1-pentene, 3-methyl-1-pentene, 1-octene, 1-decene, 1-dodecene, 1-tetradecene, 1-hexadecene, 1-octadecene, and 1-icosene; cycloolefins of 3-30, for example 3-20 carbon atoms such as, for example, cyclopentene, cycloheptene, norbornene, 5-methyl-2-norbornene, and tetracyclododecene; polar monomers: including α,β-unsaturated carboxylic acid such as acrylic acid, methacrylic acid, fumaric acid, maleic anhydride, itaconic acid, itaconic anhydride, and bicyclo[2.2.1]-5-heptene-2,3-dicarboxylic acid anhydride, and α,β-unsaturated carboxylic acid metal salts such as salts thereof of sodium, potassium, lithium, zinc, magnesium, and calcium; α,β-unsaturated carboxylic acid esters such as methyl acrylate, ethyl acrylate, n-propyl acrylate, isopropyl acrylate, n-butyl acrylate, isobutyl acrylate, t-butyl acrylate, 2-ethylhexyl acrylate, methyl methacrylate, ethyl methacrylate, n-propyl methacrylate, isopropyl methacrylate, n-butyl methacrylate, and isobutyl methacrylate; vinyl esters such as vinyl acetate, vinyl propionate, vinyl caproate, vinyl caprylate, vinyl laureate, vinyl stearate, and vinyl trifluoroacetate; and unsaturated glycidyl esters such as glycidyl acrylate, glycidyl methacrylate, and monoglycidyl itaconate.

Vinylcyclohexane, dienes, and polyenes are also useful. The diene and polyenes include cyclic or linear compounds having two or more double bonds having 4-30, such as 4-20 carbon atoms, specifically including butadiene, isoprene, 4-methyl-1,3-pentadiene, 1,3-pentadiene, 1,4-pentadiene, 1,5-hexadiene, 1,4-hexadiene, 1,3-hexadiene, 1,3-octadiene, 1,4-octadiene, 1,5-octadiene, 1,6-octadiene, 1,7-octadiene, ethylidenenorbornene, vinylnorbornene, dicyclopentadiene, 7-methyl-1,6-octadiene, 4-ethylidene-8-methyl-1,7-nonadiene, and 5,9-dimethyl-1,4,8-decatriene. Further useful are aromatic vinyl compounds including mono- or polyalkylstyrenes such as styrene, o-methylstyrene, m-methylstyrene, p-methylstyrene, o,p-dimethylstyrene, o-ethylstyrene, m-ethylstyrene, p-ethylstyrene; functional group-containing styrene derivatives such as methoxystyrene, ethoxystyrene, vinylbenzoic acid, methyl vinylbenzoate, vinylbenzyl acetate, hydroxystyrene, o-chlorostyrene, p-chlorostyrene, and divinylbenzene; 3-phenylpropylene, 4-phenylpropylene, and [alpha]-methylstyrene.

In one embodiment of the present invention the olefins may be, but are not limited to, C₂-C₃₀ α-olefins, C₂-C₃₀ functionalized alkenes, cycloalkenes, norborene and derivatives thereof, dienes, acetylenes, styrene, alkenols, alkenoic acids and derivatives or mixtures thereof. Thus, the olefins may be ethylene, propylene, butene, pentene, hexene, 4-methyl-1-pentene, octene, norborene or methacrylate. In one embodiment the olefin is ethylene or propylene. These α-olefins or functionalized alkenes may be used singly or in combination of two or more thereof.

The olefin polymerization catalyst of the present invention has a high polymerization activity, giving a polymer having a narrow molecular weight distribution, and giving an olefin copolymer having narrow composition distribution in copolymerization of two or more olefins.

The olefin polymerization catalyst of the present invention may also be used for copolymerization of an α-olefin and a conjugated diene.

The conjugated diene includes aliphatic conjugated dienes of 4-30, such as 4-20 carbon atoms. Examples of such dienes may be, but are not limited to, 1,3-butadiene, isoprene, chloroprene, 1,3-cyclohexadiene, 1,3-pentadiene, 4-methyl-1,3-pentadiene, 1,3-hexadiene, and 1,3-octdiene. These conjugate dienes may be use singly or in combination of two or more thereof.

In the present invention, in copolymerization of an α-olefin and a conjugated diene, a nonconjugated diene or a polyene may be additionally used. The nonconjugated diene and the polyene include, but is not limited to, 1,4-pentadiene, 1,5-hexadiene, 1,4-hexadiene, 1,4-octadiene, 1,5-octadiene, 1,6-octadiene, 1,7-octadiene, ethylidenenorbornene, vinylnorbornene, dicyclopentadiene, 7-methyl-1,6-octadiene, 4-ethylidene-8-methyl-1,7-nonadiene, and 5,9-dimethyl-1,4,8-decatriene.

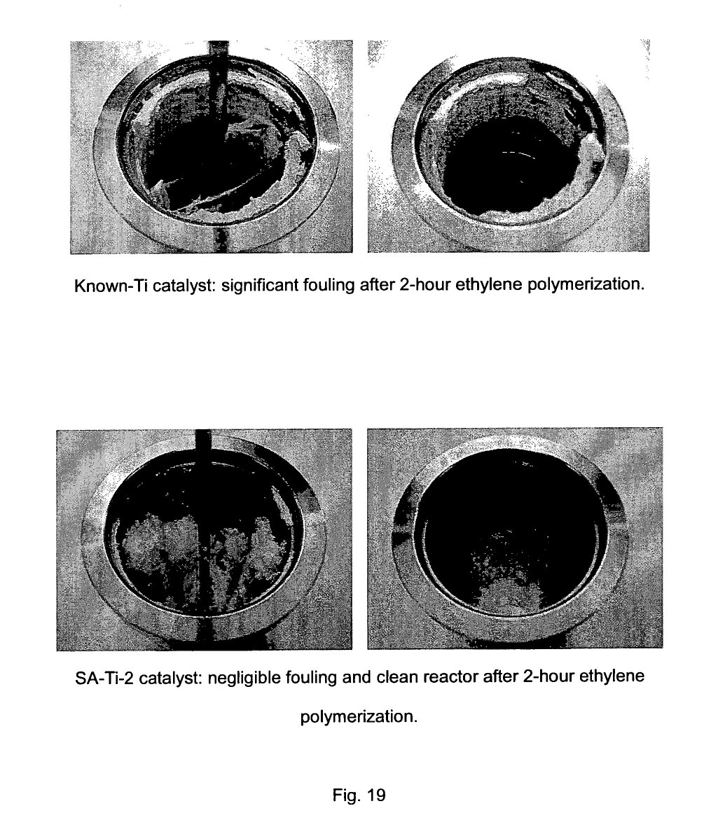

The process for producing an olefin polymer of the present invention gives the olefin polymer having a narrow molecular weight distribution at a high yield by polymerization in the presence of the above olefin polymerization catalyst. A further positive effect of the catalyst of the present invention relates to a decrease in reactor fouling. Fouling refers to the accumulation and deposition of certain material on hard surfaces. Fouling is ubiquitous and generates tremendous operational losses, not unlike corrosion. Like normal mono-nuclear catalysts, the known-Ti catalyst caused significant reactor fouling as shown in FIG. 19, hence mono-nuclear homogeneous catalyst has to be supported on a supporter for industrial applications. However, the catalyst of the present invention displayed the property of heterogeneous catalyst to prevent reactor fouling. After polymerization, the reactor was still clean.

### EXAMPLES

The following experimental examples are provided to further illustrate the present invention and are not intended to be limiting to the scope of the invention.

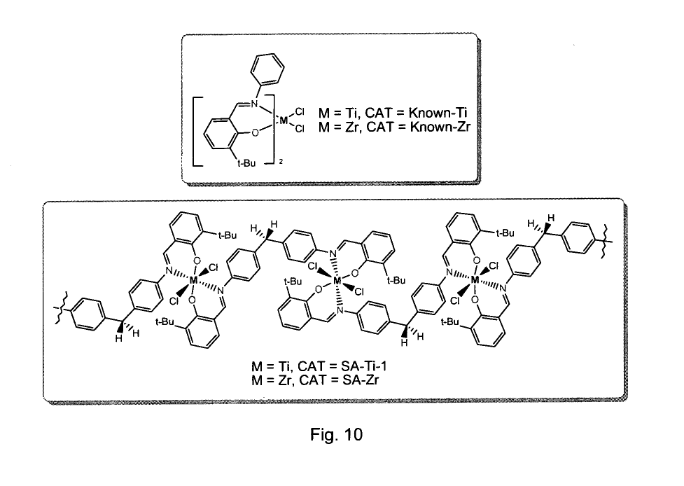

All manipulations involving air-sensitive materials were carried out by using standard Schlenk techniques or in glove box under an atmosphere of argon. 4,4′-diaminodiphenylmethane, benzidine, 3-tert-butyl-2-hydroxy-benzaldehyde and anhydrous hexane were purchased from Sigma-Aldrich and used without any pre-treatment. Methanol was dried over 4 Å molecular sieves. Dichloromethane and THF were purified using MBRAUN-SPS solvent purification system. ¹H-NMR and ¹³C-NMR were recorded in CDCl₃ on a BRUCKER 400 spectrometer. Elemental analysis was performed on a EuroEA3000 Series Elemental Analyzer. Methyl aluminoxane solution (Al %: ˜5.2%) in toluene was purchased from Chemtura Organometallics GmbH to be used directly without any pre-treatment. The known Ti and Zr catalysts based on phenoxy-imine (see FIG. 10), were prepared following the reported method^{[7]} with exactly the same procedure for the synthesis of SA-Ti-1, SA-Ti-2 and SA-Zr catalysts as below (see FIG. 9 and FIG. 15), High temperature GPC analyses of polyethylene were performed on a Polymer Labs GPC-220 with a triple detector system (refractive index, a PL-BV400 viscometer and a PD2040 dual angle light scattering detector). Typical operating conditions for analysing polyethylene are: two PLgel 10 μm Mixed B columns (300*7.5 mm) and one PLGel 10 μm guard column (50*7.5 mm) at 160° C. using 1,2,4-trichlorobenzene stabilised with 0.0125 wt. % BHT as the eluent. Polymer samples were prepared at a concentration of 1 mg/ml using a Polymer Labs SP260 sample preparation system at 150° C. until dissolved (typically about 4 to about 6 hours), followed by filtration where necessary.

### Example 1

### Preparation of bis-phenoxy-imine ligand (XVIII)

In a dried 150 ml flask, 4,4′-diaminodiphenylmethane (1.34 g, 6.76 mmol) was dissolved into 25 ml anhydrous methanol. After stirring for several minutes, 3-tert-butyl-2-hydroxy-benzaldehyde (2.65 g, 14.87 mmol) was added, followed by several drops of formic acid. The resulting mixture was stirred for one hour under room temperature and then refluxed for one day under argon ambience. After cooling to room temperature, the product was isolated by filtration, washed with 12 ml methanol and dried in vacuum, affording 3.45 g of a yellow powder product, yield 98%. ¹H-NMR (CDCl₃, 400 MHz, δ): 1.50 (s, 18H, —C(CH₃)₃), 4.04 (s, 2H, —CH₂—), 6.87-7.42 (multi, 14H, aromatic-H), 8.63 (s, 2H, —CH═N—), 13.96 (s, 2H, —OH). ¹³C-NMR (CDCl₃, 400 MHz, δ): 29.38, 34.93, 41.04, 118.34, 119.13, 121.37, 129.89, 130.30, 130.62, 137.67, 139.64, 146.66, 160.55, 162.90. Elemental analysis C₃₅H₃₈N₂O₂ (518.71): Calc.: C 81.05%, H 7.38%, N 5.40. Found: C 80.89%, H 7.41%, N 5.46%. HRMS (EI, m/z): Calculated 518.2933; Found 518.2903 (M⁺).

### Example 2

### Preparation of bis-phenoxy-imine ligand (XIX)

Bis-phenoxy-imine ligand (XIX) was synthesized with the same procedure for the synthesis of ligand (XVIII) using benzidine (1.06 g, 5.74 mmol) and 3-tert-butyl-2-hydroxy-benzaldehyde (2.09 g, 11.49 mmol) in 30 ml anhydrous methanol. Obtained 2.80 g yellow powder, yield 99%. ¹H-NMR (CDCl₃, 400 MHz, δ): 1.51 (s, 18H, —C(CH₃)₃), 6.90-7.71 (multi, 14H, aromatic-H), 8.71 (s, 2H, —CH═N—), 13.96 (s, 2H, —OH). ¹³C-NMR (CDCl₃, 400 MHz, δ): 29.36, 34.94, 118.41, 119.12, 121.74, 127.87, 130.48, 130.71, 137.72, 138.80, 147.64, 160.61, 163.11. Elemental analysis C₃₄H₃₆N₂O₂ (504.68): Calc. C 80.92%, H 7.19%, N 5.55%; Found C 80.98%, H 7.12%, N 5.62%. HRMS (EI, m/z): Calculated 504.2777; Found 504.2823 (M⁺). Single crystal was crystallized in toluene. X-Ray molecular structure was shown in FIG. 16. The crystal is monoclinic, space group C2/c. There is one molecule of C₃₄H₃₄Cl₂N₂O₂ per asymmetric unit cell. Final R values are R1=0.0536 and wR2=0.1311 for 2-theta up to 55° C.

### Example 3

### Preparation of phenoxy-imine ligand (I)

In a dried 100 ml flask, aniline (1.44 g, 15.46 mmol) was dissolved into ml anhydrous methanol under stirring. Then 3-tert-butyl-2-hydroxy-benzaldehyde (2.5 g, 14.03 mmol) was added, followed by several drops of formic acid. The resulting mixture was stirred for one hour under room temperature and then refluxed for 8 h under argon ambience. After cooling to room temperature, methanol was removed under vacuum to give a yellow residue which was purified by column chromatography eluted with hexane/ethyl acetate (10:1) affording the product as a pale yellow oil. 3.2 g, yield 90%. ¹H-NMR (CDCl₃, 400 MHz, δ): 1.54 (s, 9H, tert-Butyl), 6.91-7.48 (m, 8H, aromatic-H), 8.66 (s, 1H, —CH═N—), 13.97 (s, 1H, —OH). ¹³C-NMR (CDCl₃, 400 MHz, δ): 29.39, 34.96, 118.37, 119.10, 121.23, 126.75, 129.41, 130.39, 130.71, 137.69, 148.51, 160.58, 163.42.

### Example 4

### Synthesis of Catalyst SA-Ti-1

For accurate comparison, the catalysts SA-Ti-1, SA-Ti-2 and SA-Zr were synthesized with exactly the same procedure for the synthesis of the known Ti and Zr catalysts based on phenoxy-imine. In a dried Schlenk tube, ligand (XVIII) (1.00 g, 1.93 mmol) was dissolved into 20 ml THF. After being cooled to −78° C., 2.41 ml 1.60M n-butyllithium (3.86 mmol) hexane solution was added dropwise over a period of 10 minutes. Then the mixture was allowed to warm to room temperature and stirred for two hours. The resulting solution was added dropwise via cannula over a period of 20 minutes to a TiCl₄ (0.3657 g, 1.93 mmol)/THF (15 ml) solution under −50° C. The resulting mixture was again warmed to room temperature and stirred for 18 hours. After removal of THF, the residue solid was extracted with 30 ml dichloromethane which was then filtered to give a clear solution. Removal of dichloromethane gave a deep reddish brown solid that is the self-assembled Ti catalyst with repeating unit C₃₅H₃₆Cl₂N₂O₂Ti.xTHF which was dried in vacuum under room temperature for several hours. Elemental analysis indicated that the x is close to 1. Calculated for C₃₅H₃₆Cl₂N₂O₂Ti.THF (FW 707.61): C 66.20%, H 6.27%, N 3.96%, Ti 6.77%. Found: C 65.50%, H 6.59%, N 3.75%, Ti 5.82%. Catalyst obtained: 1.35 g, Yield 99%.

### Example 5

### Synthesis of Catalyst SA-Ti-2

The title catalyst SA-Ti-2 was synthesized with exactly the same procedure for the synthesis of SA-Ti-1 using 1.00 g ligand (XIX) (1.98 mmol) in 30 ml THF and equimolar of TiCl₄ in 30 ml THF. The product was extracted with 40 ml DCM. Removing DCM under vacuum afforded the self-assembled SA-Ti-2 catalyst as a deep reddish-brown solid with repeating unit C₃₄H₃₄Cl₂N₂O₂Ti.xTHF. Elemental analysis indicated that the x is close to 1. Calculated for C₃₄H₃₄Cl₂N₂O₂Ti.THF (FW 693.58): C 65.81%, H 6.10%, N 4.04%. Found: C 63.52%, H 5.98%, N 4.01%. Catalyst obtained: 1.37 g, Yield 99%.

### Example 6

### Synthesis of Catalyst SA-Zr

The title catalyst SA-Zr was synthesized with exactly the same procedure for the synthesis of SA-Ti-1 using 1.00 g ligand (XVIII) (1.93 mmol) and equimolar of ZrCl₄. The self-assembled Zr catalyst was obtained as pale yellow solid with repeating unit C₃₅H₃₆Cl₂N₂O₂Zr.xTHF. Elemental analysis indicated that the x is close to 1. Calculated for C₃₅H₃₆Cl₂N₂O₂Zr.THF (FW 750.93): C 62.38%, H 5.91%, N 3.73%, Zr 12.15%. Found: C 63.50%, H 6.51%, N 3.69%, Zr 10.60%. Catalyst obtained: 1.42 g, Yield 98%.

### Example 7

### Synthesis of Known Ti Catalyst Based on Phenoxy-Imine (Known-Ti)

The title Ti catalyst was synthesized with exactly the same procedure for the synthesis of SA-Ti-1 using 1.00 g ligand (I) (3.947 mmol) and equimolar of TiCl₄. The catalyst was obtained as deep reddish-brown solid with a general formula C₃₄H₃₆Cl₂N₂O₂Ti.xTHF. Elemental analysis indicated that the x is close to 1. Calculated for C₃₄H₃₆Cl₂N₂O₂Ti.THF (FW 695.59): C 65.62%, H 6.38%, N 4.03%, Ti 6.89%. Found: C 66.10%, H 6.56%, N 4.01%, Ti 6.31%. Catalyst obtained: 1.34 g, Yield 98%.

### Example 8

### Synthesis of Known Zr Catalyst Based on Phenoxy-Imine (Known-Zr)

The title catalyst was synthesized with exactly the same procedure for the synthesis of SA-Ti-1 using 1.00 g ligand (I) (3.947 mmol) and equimolar of ZrCl₄. The catalyst was obtained as yellow solid with a general formula C₃₄H₃₆Cl₂N₂O₂Zr.xTHF. Elemental analysis indicated that the x is close to 1. Calculated for C₃₄H₃₆Cl₂N₂O₂Zr.THF (FW 738.91): C 61.77%, H 6.00%, N 3.79%, Zr 12.35%. Found: C 62.62%, H 6.06%, N 3.74%, Zr 11.84%. Catalyst obtained: 1.37 g, Yield 94%.

### Example 9

### Ethylene Polymerization (General Procedure)

Polymerization was carried out in a 300 ml stainless steel autoclave equipped with a mechanical stirrer which stirring rate was adjustable. The autoclave was heated by a heating mantel. Before reaction, the autoclave was dried under vacuum at 80° C. for one hour during which period the autoclave was swept with anhydrous argon at least three times. Then the temperature was lowered to desired reaction temperature (60° C.) and the reactor was vacuumed and refilled with ethylene. Then 100 ml hexane, 2.0 mmol MAO and catalyst solution in dichloromethane were added in order with syringes under ethylene (˜10 PSI) and stirring rate 300 RPM. Then ethylene was quickly pressurized to 80 PSI (5.5 bar) and stirring rate was adjusted to 500 RPM. After the polymerization was run for the prescribed time, ethylene pressure was vented quickly and the reaction was quenched with 2 ml ethanol. Polyethylene was collected by filtration, washed with ethanol and hexane and dried in vacuum under 50° C. The obtained white polymer was weight and analyzed with GPC. Activity was calculated in unit of Kg_{PE} mol⁻¹ h⁻¹ bar⁻¹.

### (a) Catalytic Activity and Catalyst Lifetime.

Both the novel self-assembled titanium and zirconium catalysts (SA-Ti-1, SA-Ti-2 and SA-Zr) were compared with the corresponding known catalysts based on phenoxy-imine ligand (Known-Ti and Known-Zr) under practical conditions at 60° C. for up to two hours because catalyst retention time in industrial production lines is usually between 1-2 hours. The SA-Ti-1 demonstrated much higher activity than Known-Ti under different reaction time. Longer reaction time resulted in higher activity increase, up to 141% activity increase in case of 2 hours reaction, see table 1 below.

TABLE 1
Comparison of SA-Ti-1 catalyst with Known-Ti catalyst

| | Cat. | Time | PE | Activity | Mₙ | M_{w} | |
|---|---|---|---|---|---|---|---|
| Entry | (μmol) | (min) | (g) | (Kg_{PE} mol_{M}⁻¹ h⁻¹ bar⁻¹) | (×10³) | (×10³) | M_{w}/Mₙ |
| Ex. 1 | SA-Ti-1 | 30 | 6.55 | 2646.5 | 651.1 | 1656.1 | 2.5 |
| | (0.9 μmol) | | | (1.40 times, 40% increase) | | | |
| Ex. 2 | SA-Ti-1 | 60 | 11.19 | 2260.6 | 662.4 | 1904.6 | 2.9 |
| | (0.9 μmol) | | | (1.95 times, 95% increase) | | | |
| Ex. 3 | SA-Ti-1 | 120 | 15.61 | 1576.8 | 739.6 | 2391.0 | 3.2 |
| | (0.9 μmol) | | | (2.41 times, 141% increase) | | | |
| Comp. | Known-Ti | 30 | 4.69 | 1894.9 | 329.0 | 670.8 | 2.0 |
| Ex. 1 | (0.9 μmol) | | | | | | |
| Comp. | Known-Ti | 60 | 5.73 | 1157.6 | 385.0 | 768.6 | 2.0 |
| Ex. 2 | (0.9 μmol) | | | | | | |
| Comp. | Known-Ti | 120 | 6.49 | 655.6 | 493.2 | 1580.9 | 3.2 |
| Ex. 3 | (0.9 μmol) | | | | | | |

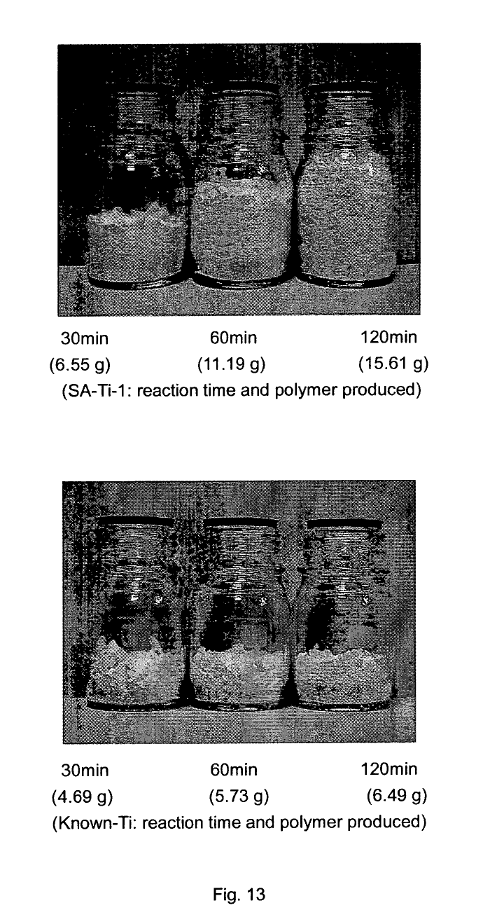

SA-Ti-1 is also more stable demonstrating much slower catalyst deactivation compared to Known-Ti. After two hours, SA-Ti-1 is still quite active, indicating a very stable long-lived robust catalyst. While the activity of Known-Ti catalyst became very low after two hours, indicating that the catalyst decomposed quickly, see table 2 below and FIG. 11. FIG. 13 clearly showed that, for SA-Ti-1 catalyst, the polyethylene increased quickly with the prolongation of reaction time, while for Known-Ti catalyst, the polyethylene increased very slowly.

TABLE 2
Comparison of activities of SA-Ti-1 and
Known-Ti in three reaction periods

| | Activity (Kg_{PE} mol_{M}⁻¹ h⁻¹ bar⁻¹) | | |
|---|---|---|---|
| Cat. | 0~30 min | 30~60 min | 60~120 min |
| SA-Ti-1 | 6.55 g | 4.64 g | 4.42 g |
| | 2646.5 | 1874.7 | 892.9 |
| | (1.40 times) | (4.46 times) | (5.82 times) |
| Known-Ti | 4.69 g | 1.04 g | 0.76 g |
| | 1894.9 | 420.2 | 153.5 |

The self-assembled SA-Zr catalyst also demonstrated much higher activity than Known-Zr catalyst under different reaction times. Longer reaction time resulted in higher activity increase up to 332% activity increase in case of 2 hours reaction, see Table 3.

TABLE 3
Comparison of SA-Zr catalyst with Known-Zr catalyst

| | Cat. | Time | PE | Activity | Mₙ | M_{w} | |
|---|---|---|---|---|---|---|---|
| Entry | (μmol) | (min) | (g) | (Kg_{PE} mol_{M}⁻¹ h⁻¹ bar⁻¹) | (×10³) | (×10³) | M_{w}/Mₙ |
| Ex. 4 | SA-Zr | 5 | 2.74 | 66424.2 | 6.77 | 238.4 | 35.2 |
| | (0.09 μmol) | | | (1.90 times, 90% increase) | | | |
| Ex. 5 | SA-Zr | 15 | 4.27 | 34505.1 | 10.28 | 421.2 | 41.0 |
| | (0.09 μmol) | | | (2.43 times, 143% increase) | | | |
| Ex. 6 | SA-Zr | 30 | 4.94 | 19959.6 | 12.03 | 569.4 | 47.3 |
| | (0.09 μmol) | | | (2.73 times, 173% increase) | | | |
| Ex. 7 | SA-Zr | 60 | 6.20 | 12525.3 | 14.23 | 796.6 | 56.0 |
| | (0.09 μmol) | | | (3.28 times, 228% increase) | | | |
| Ex. 8 | SA-Zr | 120 | 8.46 | 8545.5 | 23.37 | 900.9 | 38.6 |
| | (0.09 μmol) | | | (4.32 times, 332% increase) | | | |
| Comp. | Known-Zr | 5 | 1.44 | 34909.1 | 3.43 | 20.9 | 6.1 |
| Ex. 4 | (0.09 μmol) | | | | | | |
| Comp. | Known-Zr | 15 | 1.76 | 14222.2 | 3.63 | 37.2 | 10.2 |
| Ex. 5 | (0.09 μmol) | | | | | | |
| Comp. | Known-Zr | 30 | 1.81 | 7313.1 | 4.29 | 81.8 | 19.1 |
| Ex. 6 | (0.09 μmol) | | | | | | |
| Comp. | Known-Zr | 60 | 1.89 | 3818.2 | 4.56 | 309.3 | 67.8 |
| Ex. 7 | (0.09 μmol) | | | | | | |
| Comp. | Known-Zr | 120 | 1.96 | 1979.8 | 5.10 | 183.5 | 36.0 |
| Ex. 8 | (0.09 μmol) | | | | | | |

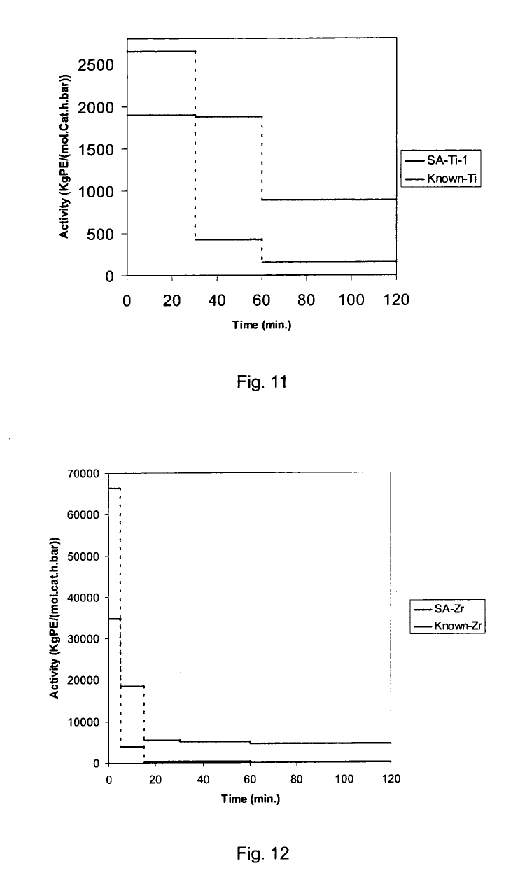

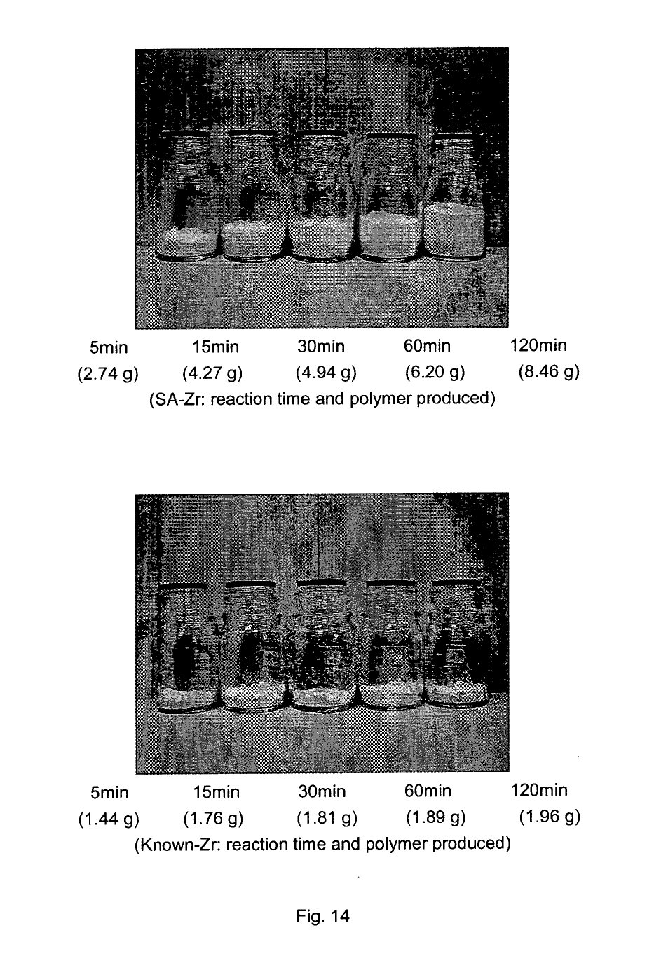

SA-Zr also demonstrated much slower catalyst deactivation than Known-Zr, see Table 4 and FIG. 12. After two hours, SA-Zr is still quite active, while Known-Zr catalyst became very weak, indicating that most of the catalysts have decomposed. FIG. 14 clearly showed that, for SA-Zr catalyst, the polyethylene increased quickly with the prolongation of reaction time, while for Known-Zr catalyst, the polyethylene looks almost the same with different reaction times.

TABLE 4
Comparison of activities of SA-Zr with Known-Zr in five reaction periods

| | Activity (Kg_{PE} mol_{M}⁻¹ h⁻¹ bar⁻¹) | | | | |
|---|---|---|---|---|---|
| Cat. | 0~5 min | 5~15 min | 15~30 min | 30~60 min | 60~120 min |
| SA-Zr | 2.74 g | 1.53 g | 0.67 g | 1.26 g | 2.26 g |
| | 66424.2 | 18545.5 | 5414.1 | 5090.9 | 4565.7 |
| | (1.90 times) | (4.78 times) | (13.40 times) | (15.75 times) | (32.29 times) |
| Known-Zr | 1.44 g | 0.32 g | 0.05 g | 0.08 g | 0.07 g |
| | 34909.1 | 3878.8 | 404.0 | 323.2 | 141.4 |

These results clearly showed that both SA-Ti-1 and SA-Zr are long-lived highly efficient ethylene polymerization catalysts, demonstrating multi-fold higher productivity compared to the known catalysts.

The SA-Ti-2 catalyst was also studied for ethylene polymerization and compared with Known-Ti catalyst under practical conditions at 60° C. for up to two hours (see Table 5). Compared to the Known-Ti catalyst, SA-Ti-2 demonstrated 52%, 125% and 208% activity increase for the three reaction times (30 min, 60 min and 120 min, respectively). It can be concluded that SA-Ti-2 catalyst also displayed much higher activity than the Known-Ti catalyst, and longer reaction time resulted in higher activity increase.

TABLE 5
Comparison of SA-Ti-2 catalyst with Known-Ti catalyst

| | Cat. | Time | PE | Activity | Mₙ | M_{w} | |
|---|---|---|---|---|---|---|---|
| Entry | (μmol) | (min) | (g) | (Kg_{PE} mol_{M}⁻¹ h⁻¹ bar⁻¹) | (×10³) | (×10³) | M_{w}/Mₙ |
| Ex. 9 | SA-Ti-2 | 30 | 7.14 | 2884.8 | 642.2 | 2878.9 | 4.5 |
| | (0.9 μmol) | | | (1.52 times, 52% increase) | | | |
| Ex. 10 | SA-Ti-2 | 60 | 12.92 | 2610.1 | 716.3 | 2933.8 | 4.1 |
| | (0.9 μmol) | | | (2.25 times, 125% increase) | | | |
| Ex. 11 | SA-Ti-2 | 120 | 20.02 | 2022.2 | 827.5 | 3011.8 | 3.7 |
| | (0.9 μmol) | | | (3.08 times, 208% increase) | | | |
| Comp. | Known-Ti | 30 | 4.69 | 1894.9 | 329.0 | 670.8 | 2.0 |
| Ex. 1 | (0.9 μmol) | | | | | | |
| Comp. | Known-Ti | 60 | 5.73 | 1157.6 | 385.0 | 768.6 | 2.0 |
| Ex. 2 | (0.9 μmol) | | | | | | |
| Comp. | Known-Ti | 120 | 6.49 | 655.6 | 493.2 | 1580.9 | 3.2 |
| Ex. 3 | (0.9 μmol) | | | | | | |

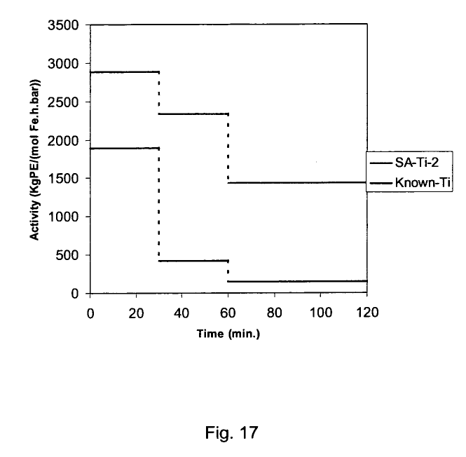

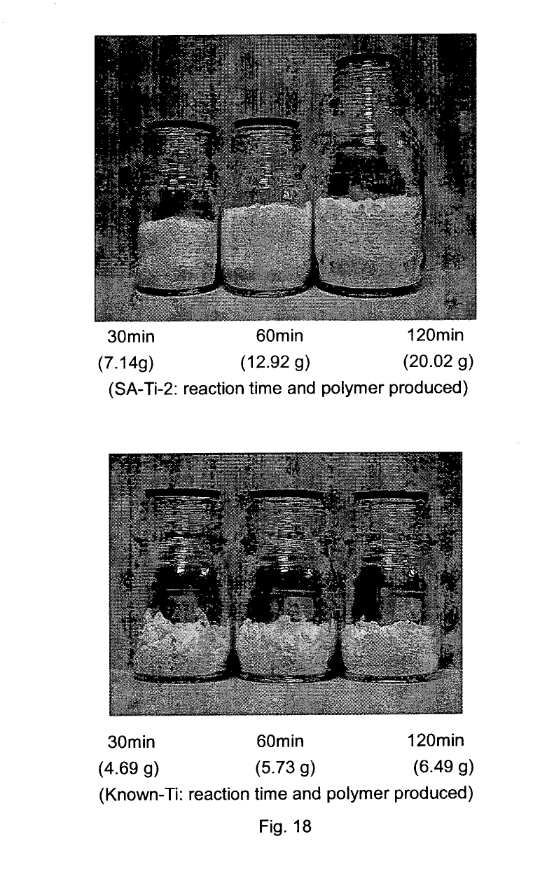

The activity of catalyst SA-Ti-2 between three reaction periods (0-30 min, 30-60 min and 60-120 min) was calculated and compared with the Known-Ti catalyst, see Table 6 and FIG. 17. Apart from displaying much higher activity, SA-Ti-2 displayed much slower catalyst deactivation indicating a long-lived robust catalyst. FIG. 18 clearly showed that, for SA-Ti-2 catalyst, the polyethylene increased quickly with the prolongation of reaction time, while for Known-Ti catalyst, the polyethylene increased very slowly.

TABLE 6
Comparison of activities of SA-Ti-2 and
Known-Ti in three reaction periods

| | Activity (Kg_{PE} mol_{M}⁻¹ h⁻¹ bar⁻¹) | | |
|---|---|---|---|
| Cat. | 0~30 min | 30~60 min | 60~120 min |
| SA-Ti-2 | 7.14 g | 5.78 g | 7.1 g |
| | 2884.8 | 2335.4 | 1434.3 |
| | (1.52 times) | (5.56 times) | (9.35 times) |
| Known-Ti | 4.69 g | 1.04 g | 0.76 g |
| | 1894.9 | 420.2 | 153.5 |

The rapid catalyst decomposition of Known-Ti and Known-Zr is predominantly caused by the transfer of supporting ligands to aluminum containing in the co-catalyst mixture (see FIG. 4). Self-assembled catalysts SA-Ti-1, SA-Ti-2 and SA-Zr have more stable structures, ligand transfer need to break the self-assembling system and this may need higher energy resulting in the ligand transfer being suppressed. Therefore, the self-assembled catalysts demonstrated much longer catalyst lifetime and much higher activity.

### (b) Molecular Weight (M_{w})

Real industry catalysts produce high molecular weight (M_{w}) polymers that are used in making final products in the markets, such as films, packing materials and tubes etc. For most of the non-cyclopentadienyl single site catalysts, one main problem is that the polymer produced has too low M_{w}. GPC analysis revealed that the novel self-assembled Ti and Zr catalysts produced much higher M_{w} PE compared to the corresponding known-Ti and known-Zr catalysts (see tables 1, 3 and 5 above). For example, for Ti catalysts with 30 min run, the molecular weight of SA-Ti-1 (Mₙ: 651.1×10³; M_{w}: 1656.1×10³) and the molecular weight of SA-Ti-2 (Mₙ: 642.2×10³; M_{w}: 2878.9×10³) are much higher than that of Known-Ti (Mₙ: 329.0×10³; M_{w}: 670.8×10³); For Zr catalysts with 2 h run, the molecular weight of SA-Zr (Mₙ: 23.37×10³; M_{w}: 900.9×10³) is also much higher than that of Known-Zr (Mₙ: 5.10×10³; M_{w}: 183.5×10³).

It is very useful and interesting that SA-Ti-1 and SA-Ti-2 catalyst can produce Ultra High Molecular Weight PE of more than one million with narrow molecular weight distribution that is difficult to be produced by traditional Ziegler-Natta catalysts and most of the metallocene and non-cyclopentadienyl single site homogeneous catalysts under practical conditions in high activity. SA-Ti-1 produced PE with M_{w} up to 1.656 millions even with 30 min run under low pressure (5.5 bar). With 2 h run, the M_{w} is high up to 2.391 millions. Furthermore the PE produced has narrow molecular weight distribution (M_{w}/Mₙ=2.5-3.2). SA-Ti-2 produced PE with M_{w} up to 2.879 millions even with 30 min run under low pressure (5.5 bar). With 2 h run, the M_{w} is high up to 3.012 millions. Furthermore the PE produced still has relatively narrow molecular weight distribution (M_{w}/Mₙ=3.7-4.5). Ultra High Molecular Weight PE is a kind of very useful material with a broad range of applications, such as Connecting Straps, Rollers, Gears, Gear Wheels etc. These results clearly indicated that SA-Ti-1 and SA-Ti-2 can produce high quality PE under low pressure with high activity.

### (c) Reactor Fouling

Like normal mono-nuclear catalysts, the known-Ti catalyst caused significant reactor fouling as shown in FIG. 19, hence mono-nuclear homogeneous catalyst has to be supported on a supporter for industrial applications. Besides much higher activity, much slower catalyst deactivation and producing much higher MW polymer, the catalyst SA-Ti-2 displayed the property of heterogeneous catalyst to prevent reactor fouling. After polymerization, the reactor was still clean as shown in FIG. 19. Anti-fouling property is very important for achieving continuous production in industry.

### REFERENCES

The following references are cited in the present application:
1. a) T. J. Pullukat, and R. E. Hoff, Catal. Rev.-Sci. Eng. 1999, 41, 389-428. b) H.-K. Luo, R.-G. Tang, K.-J. Gao, J. Cat. 2002, 210, 328-339. c) N. Kashiwa, T. Tokuzumi, H. Fujimura, U.S. Pat. No. 3,642,746 (1972). d) H.-K. Luo, R.-G. Tang, K.-J. Gao, Q.-F. Zhao, J.-Y. An, H. Yang, J.-S. Huo, WO 02/26838 (2002). e) H.-K. Luo, R.-G. Tang, H. Yang, Q.-F. Zhao, WO 02/48207 (2002).
2. a) H. Sinn, W. Kaminsky, H.-J. Vollmer, and R. Woldt, Angew. Chem. Int. Ed. 1980, 19, 390-392. b) H. H. Brintzinger, D. Fischer, R. Muelhaupt, B. Rieger, and R. M. Waymouth, Angew. Chem. Int. Ed. 1995, 34, 1143-1170. c) W. Kaminsky, J. Chem. Soc., Dalton Trans. 1998, 1413-1418. d) W. Kaminsky, J. Polym. Sci.: Part A: Polym. Chem. 2004, 42, 3911-3921. e) Exxon Chemical Patents Inc. WO 96/00243 (1996); WO 96/11960 (1996).
3. a) G. J. P. Britovsek, V. C. Gibson, and D. F. Wass, Angew. Chem. Int. Ed. 1999, 38, 428-447. b) S. D. MeI, L. K. Johnson, M. Brookhart, Chem. Rev. 2000, 100, 1169-1203. c) V. C. Gibson and S. K. Spitzmesser, Chem. Rev. 2003, 103, 283-315.
4. a) L. K. Johnson, C. M. Killian, M. Brookhart, J. Am. Chem. Soc. 1995, 117, 6414-6415. b) M. D. Leatherman, S. A. Svejda, L. K. Johnson, M. Brookhart, J. Am. Chem. Soc. 2003, 125, 3068-3081. c) C. M. Killian, D. J. Tempel, L. K. Johnson, M. Brookhart, J. Am. Chem. Soc. 1996, 118, 11664-11665. d) D. P. Gates, S. A. Svejda, E. O^{˜}nate, C. M. Killian, L. K. Johnson, P. S. White, M. Brookhart, Macromolecules 2000, 33, 2320-2334. e) L. K. Johnson, C. M. Killian, S. D. Arthur, J. Feldman, E. F. McCord, S. J. McLain, K. A. Kreutzer, M. A. Bennett, E. B. Coughlin, S. D. Ittel, A. Parthasarathy, D. J. Tempel, M. Brookhart, WO 96/23010 (1996).
5. a) B. L. Small, M. Brookhart, A. M. A. Bennett, J. Am. Chem. Soc. 1998, 120, 4049-4050. b) A. M. A. Bennett, WO 98/27124 (1998). c) G. J. P. Britovsek, V. C. Gibson, B. S. Kimberley, P. J. Maddox, S. J. McTavish, G. A. Solan, A. J. P. White and D. J. Williams, Chem. Commun. 1998, 849-850. d) G. J. P. Britovsek, B. A. Dorer, V. C. Gibson, B. S. Kimberley, G. A. Solan, WO 99/12981 (1999). e) G. J. P. Britovsek, V. C. Gibson, O. D. Hoarau, S. K. Spitzmesser, A. J. P. White, and D. J. Williams, Inorg. Chem. 2003, 42, 3454-3465. f) G. J. P. Britovsek, M. Bruce, V. C. Gibson, B. S. Kimberley, P. J. Maddox, S. Mastroianni, S. J. McTavish, C. Redshaw, G. A. Solan, S. Stroemberg, A. J. P. White, and D. J. Williams, J. Am. Chem. Soc. 1999, 121, 8728-8740. g) B. L. Small, M. Brookhart, Macromolecules 1999, 32, 2120-2130.
6. a) T. R. Younkin, E. F. Connor, J. I. Hendreson, S. K. Friedrich, R. H. Grubbs, D. A. Bansleben, Science 2000, 287, 460-462; b) C. Wang, S. K. Friedrich, T. R. Younkin, R. T. Li, R. H. Grubbs, D. A. Bansleben, M. W. Day, Organometallics 1998, 17, 3149-3151; c) D. A. Bansleben, S. K. Friedrich, T. R. Younkin, R. H. Grubbs, C. Wang, R. T. Li, WO 98/42664 (1998); d) L. K. Johnson, A. M. A. Bennett, S. D. Ittel, L. Wang, A. Parthasarathy, E. Hauptman, R. D. Simpson, J. Feldman, E. B. Coughlin, WO 98/30609 (1998).
7. a) S. Matsui, K. Tsuru, M. Nitabaru, T. Fujita, M. Mitani, Y. Takagi, T. Nakano, EP 0 950 667 (1999). b) N. Matsukawa, EP 1 013 674 (1999). c) T. Fujita, Y. Tohi, M. Mitani, S. Matsui, J. Saito, M. Nitabaru, K. Sugi, H. Makio, T. Tsutsui, U.S. Pat. No. 6,875,718 (2005). d) Y. Nakayama, H. Kaneko, H. Bandoh, J. Saito, S. Kojoh, M. Mitani, Y. Suzuki, S. Matsui, N. Kashiwa, T. Fujita, U.S. Pat. No. 6,897,176 (2005). e) Y. Inoue, S. Matsui, Y. Suzuki, Y. Yoshida, Y. Takagi, T. Fujita, T. Matsugi, T. Nakano, U.S. Pat. No. 7,053,159 (2006). f) S. Ishii, M. Mitani, J. Saito, S. Matsuura, N. Matsukawa, K. Tsuru, T. Fujita, U.S. Pat. No. 7,109,284 (2006). g) S. Matsui, M. Mitani, J. Saito, Y. Tohi, H. Makio, N. Matsukawa, Y. Takagi, K. Tsuru, M. Nitabaru, T. Nakano, H. Tanaka, N. Kashiwa, and T. Fujita, J. Am. Chem. Soc. 2001, 123, 6847-6856. h). S. Ishii, M. Mitani, J. Saito, S. Matsuura, S. Kojoh, N. Kashiwa, and T. Fujita, Chem. Lett. 2002, 740˜741. i). S. Matsui, Y. Tohi, M. Mitani, J. Saito, H. Makio, H. Tanaka, M. Nitabaru, T. Nakano and T. Fujita, Chem. Lett. 1999, 1065-1066. j). S. Ishii, M. Mitani, J. Saito, S. Matsuura, R. Furuyama and T. Fujita, Sci. Tech. Cat. 2002, 49-54.
8. a) P. Woodman, P. B. Hitchcock, P. Scott, Chem. Commun. 1996, 2735-2736. b) P. D. Knight, A. J. Clarke, B. S. Kimberley, R. A. Jackson, P. Scott, Chem. Commun. 2002, 352-353. c) P. D. Knight, G. Clarkson, M. L. Hammond, B. S. Kimberley, P. Scott, J. Organomet. Chem. 2005, 690, 5125˜5144. d) G. J. Clarkson, V. C. Gibson, P. K. Y. Goh, M. L. Hammond, P. D. Knight, P. Scott, T. M. Smit, A. J. P. White and D. J. Williams, Dalton Trans. 2006, 5484-5491.
9. a) M. Heitbaum, F. Glorius, and I. Escher, Angew. Chem. Int. Ed. 2006, 45, 4732-4762. b) X. Wang, K. Ding, J. Am. Chem. Soc. 2004, 126, 10524-10525. c) L. Shi, X. Wang, C. A. Sandoval, M. Li, Q. Qi, Z. Li, and K. Ding, Angew. Chem. Int. Ed. 2006, 45, 4108-4112.

1-48. (canceled)

49. A self-assembled olefin polymerization catalyst having a linear or macrocyclic structure, comprising a transition metal compound according to formula (I)
L_{q}MₘXₙ (I)
wherein
M is a transition metal selected from the group consisting of Ti, Zr, Hf, V, Nb, Ta, Sm, Yb and mixtures thereof;
X is independently selected from the group consisting of H, halogen, CN, optionally substituted N(R^{a})₂, OH, optionally substituted C₁-C₂₀ alkyl, optionally substituted C₁-C₂₀ alkoxy, wherein R^{a} is independently selected from the group consisting of optionally substituted C₁-C₂₀ alkyl, optionally substituted C₆-C₂₀ aryl and halogen;
q is an integer of at least 2;
m is an integer of at least 2;
n is an integer making (I) electrically neutral;
L is independently a ligand which has at least two linked coordination units,
wherein each coordination unit binds to a different transition metal atom, and
wherein said ligand L has the following formula (II) 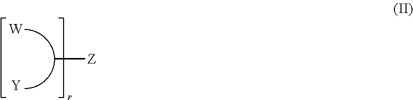
wherein
each WY unit forms a coordination unit, wherein WY is 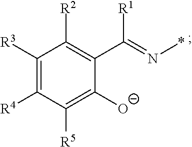
r is an integer of at least 2; Z is a bridging spacer selected from the group consisting of bis-linkers, tri-linkers, tetrakis-linkers, multi-linkers having five or more than five linking sites, and macro polymeric multi-linkers, wherein Z has a size, length and angle so that each coordination units WY binds to a different transition metal; and wherein the his-linker is selected from the group consisting of 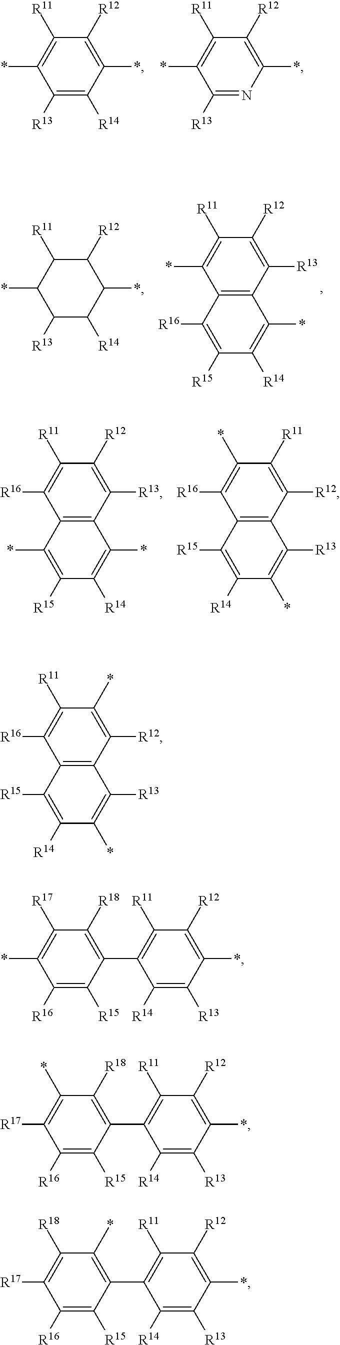 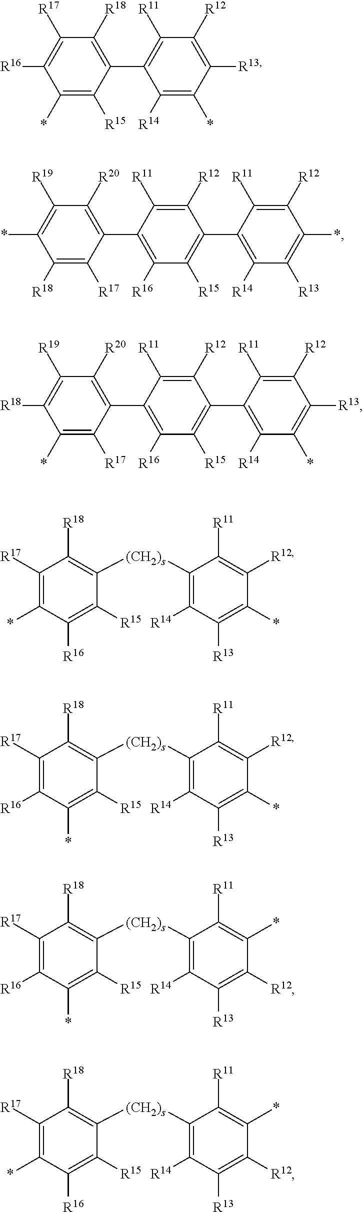 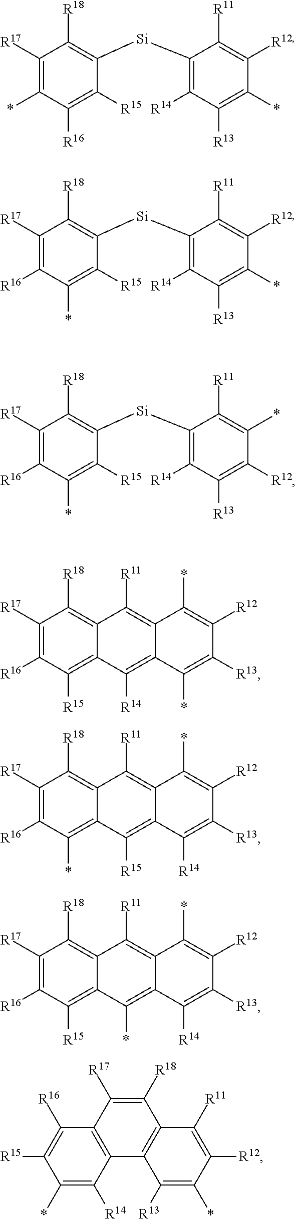 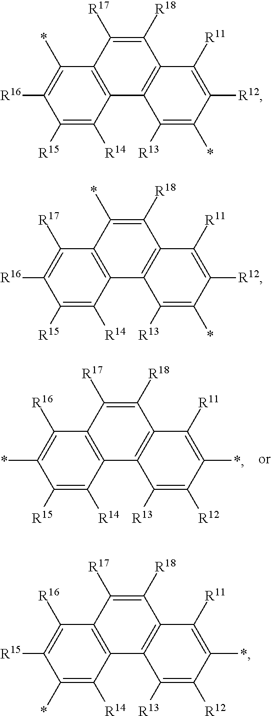
wherein the tri-linker is selected from the group consisting of 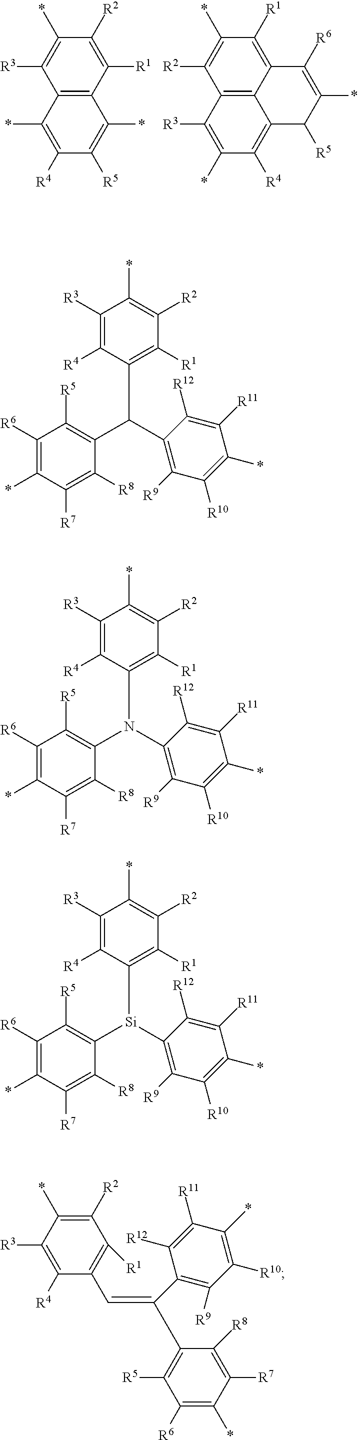
wherein the tetrakis-linker is selected from the group consisting of 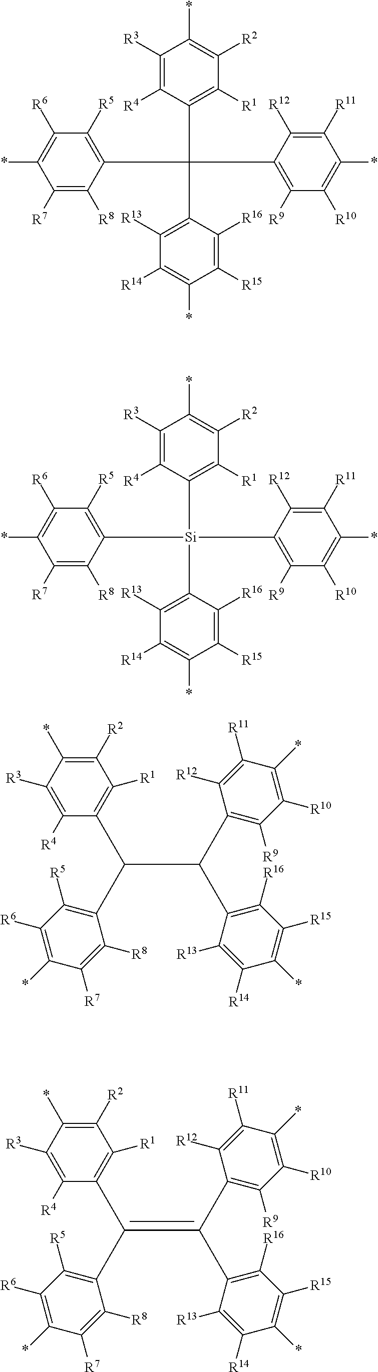 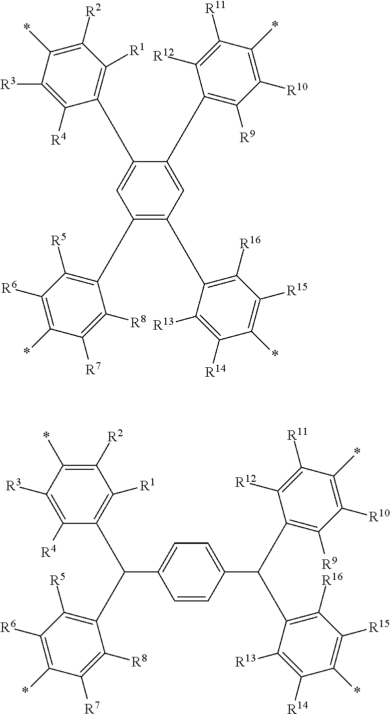
wherein R¹ to R²⁰ may be the same or different and are each selected from the group consisting of H, optionally substituted straight-chain or branched C₁-C₂₀ alkyl, optionally substituted straight-chain or branched C₂-C₂₀ alkenyl, optionally substituted straight-chain or branched C₂-C₂₀ alkynyl, optionally substituted C₆-C₂₀ aryl, optionally substituted C₆-C₂₀ heteroaryl, halogen, OH, NO₂, and CN, wherein two or more of R¹ to R⁷ may be bonded to each other to form a ring, and s is an integer from 1 to 20.

50. The self-assembled olefin polymerization catalyst according to claim 49, wherein the ligand L is selected from the group consisting of

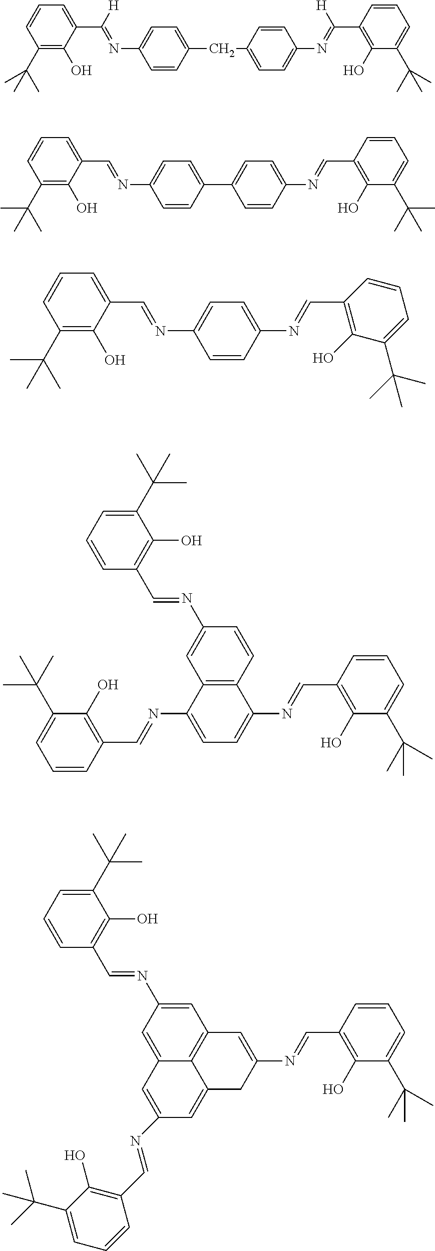

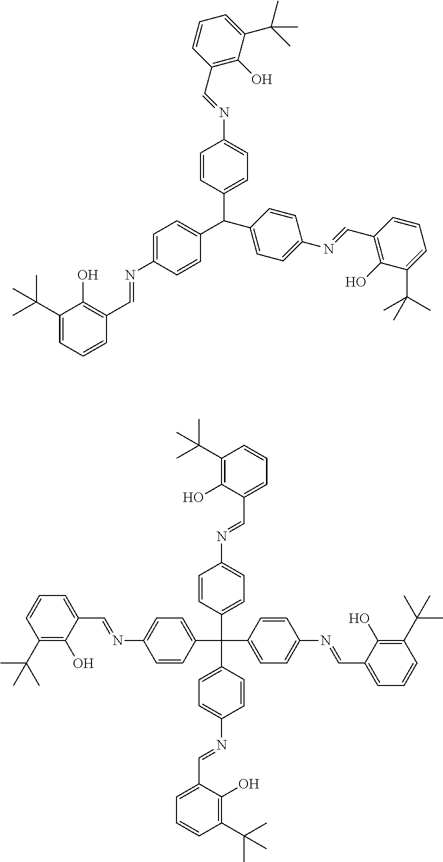

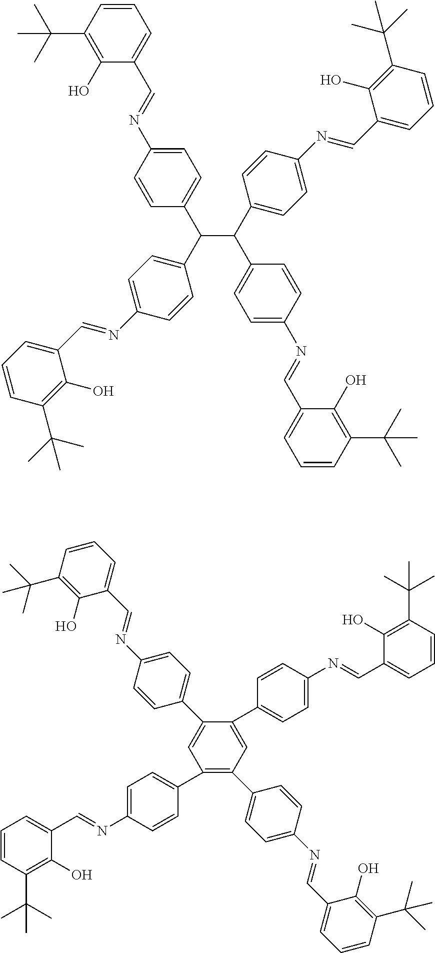

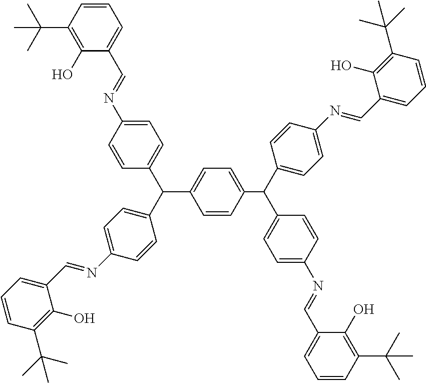

51. The self-assembled olefin polymerization catalyst according to claim 49, wherein the molar ratio of coordination unit WY to metal is about 0.5:1 to about 6:1.

52. The self-assembled olefin polymerization catalyst according to claim 51, wherein the molar ratio of coordination unit WY to metal is about 1:1 to about 3:1.

53. The self-assembled olefin polymerization catalyst according to claim 49, wherein the transition metals are selected from the group consisting of Ti, Zr and mixtures thereof.

54. The self-assembled olefin polymerization catalyst according to claim 49, wherein X is selected from the group consisting of F, Cl, Br, I, H, CH₃, CH₂CH₃, OCH₃, OCH₂CH₃, OCH(CH₃)₃, OC(CH₃)₃, OC₆H₆, CN, N(CH₃)₂, and N(CH₂CH₃)₂.

55. The self-assembled olefin polymerization catalyst according to claim 49, wherein the catalyst is a homogeneous or heterogeneous catalyst.

56. The self-assembled olefin polymerization catalyst according to claim 49, further comprising a solid support.

57. The self-assembled olefin polymerization catalyst according to claim 56, wherein the solid support is an inorganic material or an organic material.

58. The self-assembled olefin polymerization catalyst according to claim 57, wherein the solid support is an inorganic material selected from the group consisting of silica, alumina, titania, magnesium chloride, and mixtures thereof.

59. The self-assembled olefin polymerization catalyst according to claim 49, wherein the catalyst forms a 3-Dimensional organometallic framework.

60. The self-assembled olefin polymerization catalyst according to claim 49, wherein the catalyst forms a macrocyclic assembling structure containing at least two metal centres.

61. The self-assembled olefin polymerization catalyst according to claim 49 further comprising at least one co-catalyst selected from the group consisting of an organometallic compound, an organoaluminum oxy-compound, and an ionizing ionic compound.

62. The self-assembled olefin polymerization catalyst according to claim 61, wherein the co-catalyst is a conventional methyl aluminoxane (MAO), a modified methyl aluminoxane (MMAO), a metal salt of (C₆F₅)₄B⁻ and a combination of i-BuₘAl(OR)ₙ with MgCl₂.

63. A process for polymerization or copolymerization of an olefin or a mixture of olefins in the presence of the self-assembled olefin polymerization catalyst according to claim 49.

64. The process according to claim 63, wherein the process is carried out at a pressure in the range of about 0.1 MPa to about 10 MPa.

65. The process according to claim 63, wherein the process is carried out in a temperature range of about −50° C. to about 150° C.

66. The process according to claim 63, wherein the process is carried out at a catalyst:co-catalyst mole ratio of about 1:1 to about 1:5000.

67. The process according to claim 66, wherein the process is carried out at a catalyst:co-catalyst mole ratio of about 1:1 to about 1:2000.

68. The process according to claim 63, wherein the olefin is selected from the group consisting of C₂-C₃₀ α-olefins, C₂-C₃₀ functionalized alkenes, cycloalkenes, norborene and derivatives thereof, dienes, acetylenes, styrene, alkenols, alkenoic acids and derivatives or mixtures thereof.

69. The process according to claim 68, wherein the olefins are selected from the group consisting of ethylene, propylene, butene, pentene, hexene, octene, norborene and methacrylate.

70. The process according to claim 69, wherein the olefin is ethylene and propylene.

71. The process according to claim 49, wherein Z is a bis-linker selected from 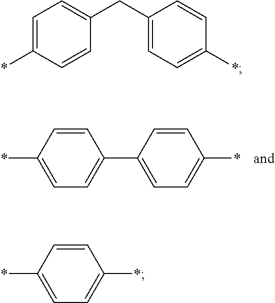
R¹ to R⁴ are all hydrogen; R⁵ is t-butyl and X is selected from halogen.

72. Polyolefins obtained according to the process of claim 63.

73. Polyolefins according to claim 72 having a molecular weight in the range from low molecular weight polyolefins to ultra high molecular weight polyolefins.

74. A compound according to the following formula (II) 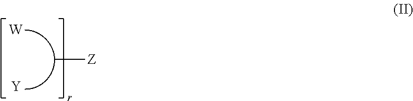
wherein
each WY unit forms a coordination unit, wherein WY is 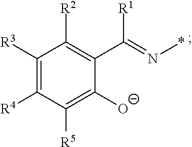
r is an integer of at least 2;
Z is a bridging spacer selected from the group consisting of bis-linkers, tri-linkers, tetrakis-linkers, multi-linkers having five or more than five linking sites, and macro polymeric multi-linkers, wherein Z has a size, length and angle so that each coordination unit WY may bind to different transition metal atom; and wherein the bis-linker is selected from the group consisting of, 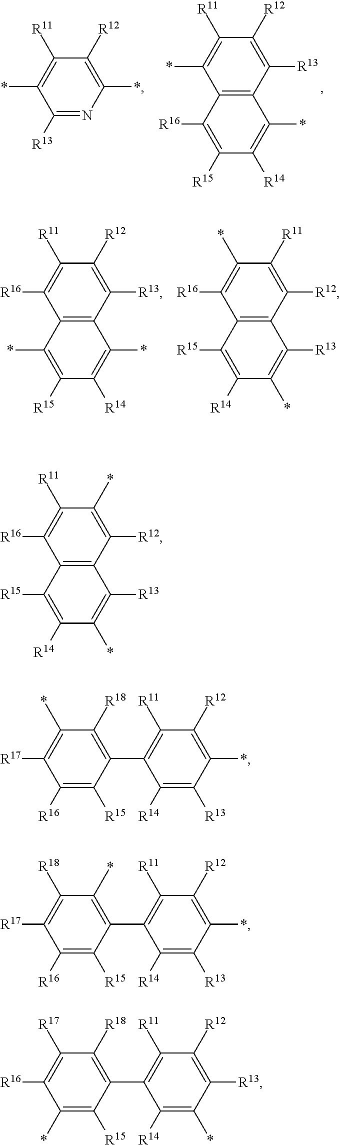 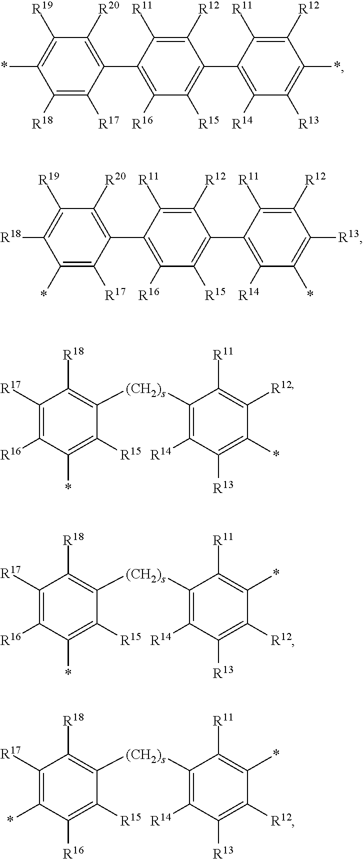 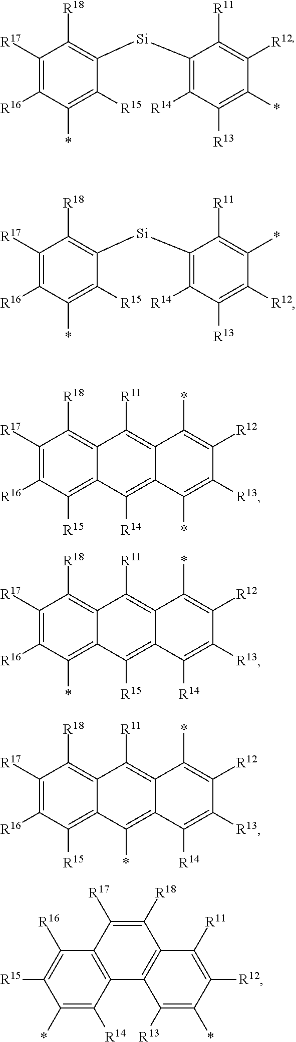 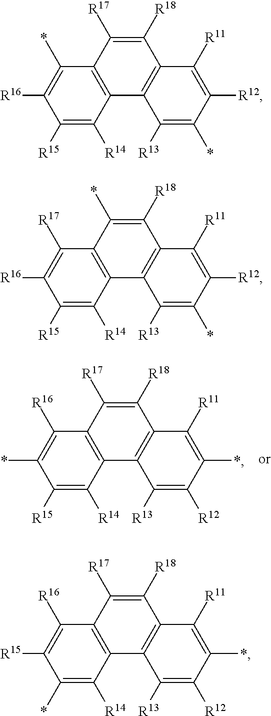
wherein the tri-linker is selected from the group consisting of 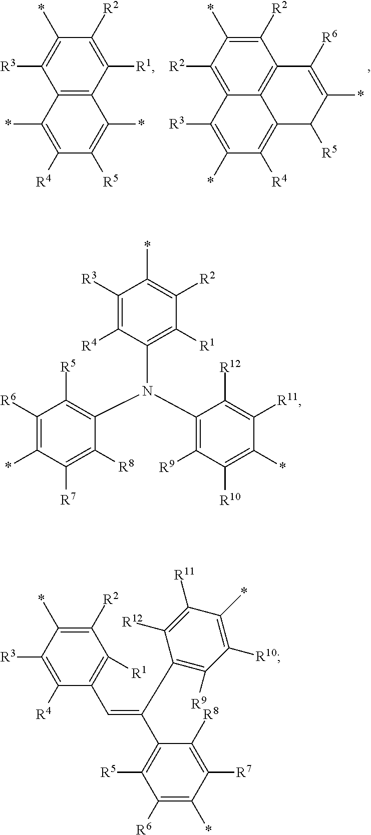
wherein the tetrakis-linker is selected from the group consisting of 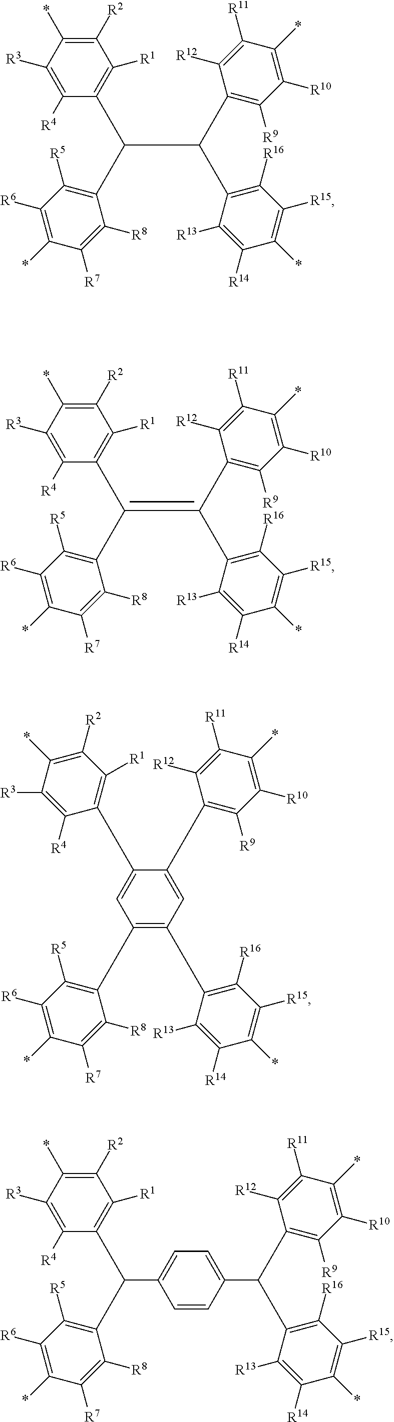
wherein R¹ to R²⁰ may be the same or different and are each selected from the group consisting of H, optionally substituted straight-chain or branched C₁-C₂₀ alkyl, optionally substituted straight-chain or branched C₂-C₂₀ alkenyl, optionally substituted straight-chain or branched C₂-C₂₀ alkynyl, optionally substituted C₆-C₂₀ aryl, optionally substituted C₆-C₂₀ heteroaryl, halogen, OH, NO₂, and CN, wherein two or more of R¹ to R⁷ may be bonded to each other to form a ring; and s is an integer from 1 to 20.

75. The compound according to claim 74, wherein Z is a bis-linker selected from 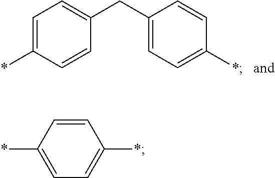
R¹ to R⁴ are all hydrogen and R⁵ is t-butyl.

76. A process for producing the compound according to claim 74 by Schiff-Base condensation between an aldehyde or ketone with an di-aniline, tri-aniline or tetrakis-aniline.

77. The process according to claim 76, wherein the aldehyde or ketone is 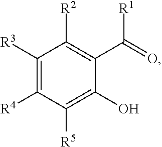
wherein R¹ to R⁵ are as described in claim 49.

78. The process according to claim 76, wherein the di-aniline, tri-aniline or tetrakis-aniline is selected from the group consisting of 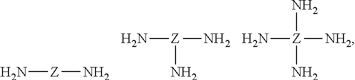
wherein Z is as described in claim 49.

79. A process for producing the compound according to claim 74, by Schiff-Base condensation between an aniline and an di-aldehyde/di-ketone, tri-aldehyde/tri-ketone or tetrakis-aldehyde/tetrakis-ketone.

80. The process according to claim 79, wherein the aniline is selected from the group consisting of 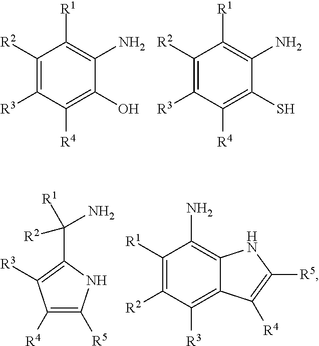
wherein R¹ to R⁵ are as described in claim 49.

81. The process according to claim 79, wherein the di-aldehyde/di-ketone, tri-aldehyde/tri-ketone or tetrakis-aldehyde/tetrakis-ketone is selected from the group consisting of 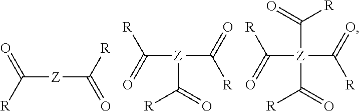
wherein R and Z are as described in claim 49.

82. The process for producing the compound according to any of claim 76, wherein the Schiff-Base condensation may be promoted by an acid catalyst selected from the group consisting of formic acid, acetic acid, p-toluenesulfonic acid, Lewis acid and a solid catalyst.
